SEQUENCE LISTING (1 sequence)

SEQ ID NO: 1 - protein, 40 aa, synthetic construct
  REGION 1..40: note = Description of sequence: peptide, specifically a MANP peptide
SLRRSSCFGGRMDRIGAQSGLGCNSFRYRITAREDKQGWA

(19) United States
(12) Patent Application Publication
(10) Pub. No.: US 2026/0295012 A1
(43) Pub. Date: Oct. 1, 2026

(54) Methods of Lowering Blood Pressure Using Fixed Doses of MANP

(71) Applicant: E-Star Biotech, LLC, New York, NY (US)

(72) Inventor: Jesse W. CROWNE, Salt Lake City, UT (US)

(73) Assignee: E-Star Biotech, LLC, New York, NY (US)

(21) Appl. No.: 19/635,463

(22) Filed: Mar. 31, 2026

Related U.S. Application Data
(60) Provisional application No. 63/781,757, filed on Apr. 1, 2025.

(51) Int. Cl. A61K 38/22; A61P 9/12

(52) U.S. Cl. CPC A61K 38/2242; A61P 9/12

(57) ABSTRACT

This disclosure provides methods and compositions for treating resistant hypertension in a patient in need thereof with fixed doses. This disclosure provides methods and compositions for lowering systolic blood pressure in a patient in need thereof with fixed doses.

### CROSS-REFERENCE TO RELATED APPLICATION

This application claims the benefit of U.S. Provisional Application No. 63/781,757, filed Apr. 1, 2025, which is incorporated by reference herein in its entirety.

### INCORPORATION OF SEQUENCE LISTING

A sequence listing contained in the file named “P35678WO00_SL.xml” which is 2,232 bytes (measured in operating system MS-Windows®), created on Mar. 7, 2026, containing a total number of one sequence, (SEQ ID NO:1), is filed electronically herewith and incorporated by reference in its entirety.

### BACKGROUND OF THE INVENTION

Hypertension, also known as high blood pressure (BP), is a long-term medical condition in which BP in the arteries is persistently elevated. While it usually does not cause symptoms, it is a major risk factor for stroke, coronary artery disease, heart failure, atrial fibrillation, peripheral arterial disease, vision loss, chronic kidney disease, and dementia. Difficult-to-control/Resistant Hypertension (DTC/RH) is a condition where blood pressure remains elevated despite optimal use of three antihypertensive medications of different classes, including a diuretic. Such patients are more likely to have a secondary cause and to suffer end-organ damage. DTC/RH remains challenging to treat and with no approved drugs or devices.

Atrial natriuretic peptide (ANP) plays a key role in BP homeostasis. Specifically, ANP exerts a number of cardiorenal and metabolic actions through the particulate guanylyl cyclase A receptor (GC-A) and its second messenger 3′,5′-cyclic guanosine monophosphate (cGMP). In humans, higher levels of circulating ANP is associated with lower BP and protection from hypertension. Although ANP has been regarded as a promising agent for treating hypertension, its rapid enzymatic degradation and the need for continuous intravenous infusion have limited its clinical development. MANP is a novel atrial natriuretic peptide (ANP) analogue engineered to overcome the therapeutic limitations of native ANP.

Obesity induces upregulation of peroxisome proliferator-activated receptor gamma (PPARγ), which mediates expression of the natriuretic peptide receptor C (NPR-C) in adipocytes. Both ANP and MANP bind to NPR-C, and are cleared from circulation once bound. Due to increased NPR-C expression in obese individuals, cGMP response to endogenous MANP is expected to be related to MANP administered per kilogram of body weight. As such, previous studies of the effects of MANP in animal models and human patients were carried out using weight-based dosing. Additionally, dosing of drugs administered via subcutaneous injection, like insulin or blood thinners, are generally calculated by weight. Examples of drugs used in hypertension that may be dosed by weight include hydralazine, heparins, diltiazem, and insulin.

### SUMMARY

Without being limited by theory, the subject matter of the present disclosure includes, in part, on the surprising changes in cGMP with fixed doses of MANP administered to patients who are in need of lowering blood pressure, or who have resistant hypertension. Again, without being limited by theory, the subject matter of the present disclosure includes, in part on the surprising development that administering MANP to patients causes a reduction in systolic blood pressure in patients with a baseline or initial blood pressure of greater than 140 mmHg, but not in patients with a baseline or initial blood pressure of less than 140 mmHg.

The present disclosure provides a method of treating resistant hypertension in a patient in need thereof, comprising administering to the patient about 300 mcg of MANP.

The present disclosure provides a method of treating resistant hypertension in a patient in need thereof, comprising administering to the patient about 450 mcg of MANP.

The present disclosure provides a method of treating resistant hypertension in a patient in need thereof, comprising administering to the patient about 600 mcg of MANP.

The present disclosure provides a method of lowering systolic blood pressure in a patient in need thereof, comprising administering to the patient about 300 mcg of MANP.

The present disclosure provides a method of lowering systolic blood pressure in a patient in need thereof, comprising administering to the patient about 450 mcg of MANP.

The present disclosure provides a method of lowering systolic blood pressure in a patient in need thereof, comprising administering to the patient about 600 mcg of MANP.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1 depicts the sitting systolic blood pressure of study patients over the duration of the study.

FIG. 2 depicts the change from baseline sitting systolic blood pressure of study patients over the duration of the study.

FIG. 3 depicts the mean cGMP levels of study patients.

FIG. 4 depicts the change in cGMP levels as a function of the weight-based dose of MANP.

FIG. 5 depicts the total dose received by each patient at each dose level.

FIG. 6 depicts that the change in cGMP levels is correlated to the total dose of MANP.

FIG. 7A depicts that the change in cGMP is more correlated to the total amount of MANP received for patients with SBP≥140 mmHg.

FIG. 7B depicts the change in cGMP is less correlated to the weight-based dose of MANP received for patients with SBP≥140 mmHg.

FIG. 8A depicts the change in cGMP and the total amount of MANP received for patients with SBP<140 mmHg.

FIG. 8B depicts the change in cGMP and the weight-based dose of MANP received for patients with SBP<140 mmHg.

### DETAILED DESCRIPTION

The present disclosure describes methods of lowering blood pressure and methods for sustained lowering of blood pressure in a patient in need thereof, comprising administering one or more fixed doses of MANP.

This description is not intended to be a detailed catalog of all the different ways in which the disclosure may be implemented, or all the features that may be added to the instant disclosure. For example, features illustrated with respect to one embodiment may be incorporated into other embodiment, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the subject matter of the present disclosure. It is intended that no part of this specification be construed to effect a disavowal of any part of the full scope of the invention. Hence, the following descriptions are intended to illustrate some particular embodiments of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art. Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps and/or actions may be modified without departing from the scope of the present disclosure.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a measurement value and the like, is meant to encompass variations of ±20%, ±10%, ±5%, ±1%, ±0.5%, or even±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “exemplary” is used to mean serving as an example, instance, or illustration. Any embodiment or aspect described as “exemplary” is not necessarily to be construed as preferred or advantageous over other embodiments or aspects, nor is it meant to preclude equivalent structures and techniques known to those of ordinary skill in the art. Rather, use of the word exemplary is intended to present concepts in a concrete fashion, and the disclosed subject matter is not limited by such examples.

### Methods for Lowering Blood Pressure Using Fixed Doses

In an aspect, the present disclosure provides for, and includes, a method of treating resistant hypertension in a patient in need thereof, comprising administering to the patient a fixed dose of MANP. In an aspect, a fixed dose is 300 mcg of MANP. In an aspect, a fixed dose is 450 mcg of MANP. In an aspect, a fixed dose is 600 mcg of MANP.

As used herein, the term “treat” or “treatment” is an approach for obtaining beneficial or desired clinical results. In an aspect, the term “treat” or “treatment” means to administer MANP as disclosed herein that partially or completely alleviate, ameliorate, relieve, inhibit, reduce severity of, and/or reduce incidence of one or more symptoms, features, or causes of resistant hypertension. In an aspect, a symptom of resistant hypertension is elevated systolic blood pressure. In an aspect, a symptom of resistant hypertension is elevated diastolic blood pressure. In an aspect, a symptom of resistant hypertension is reduced cGMP levels. In an aspect, a symptom of resistant hypertension is dysregulated cGMP levels.

In an aspect, the present disclosure provides for, and includes, a method of lowering systolic blood pressure in a patient in need thereof, comprising administering to the patient a fixed dose of MANP. In an aspect, a fixed dose is 300 mcg of MANP. In an aspect, a fixed dose is 450 mcg of MANP. In an aspect, a fixed dose is 600 mcg of MANP.

### Blood Pressure and Measurement

As used herein, “blood pressure” refers to the pressure of circulating blood against the walls of blood vessels. In an aspect, blood pressure is the pressure in an artery. In an aspect, blood pressure is the pressure in a vein. In an aspect, blood pressure is the pressure in a brachial artery. In an aspect, blood pressure is the mean arterial pressure. In an aspect, blood pressure is a pulse pressure. In an aspect, blood pressure is measured in millimeters of mercury (mmHg).

As used herein, “systolic blood pressure” refers to the maximum pressure during one heartbeat or cardiac cycle. As used here, “diastolic blood pressure” refers to the minimum pressure between two heartbeats in a cardiac cycle.

In an aspect, BP is measured by methods known in the art. In an aspect, BP is measured by direct or invasive methods. In an aspect, BP is measured by insertion of a cannula into the artery. In an aspect, the cannula is connected to an electronic pressure transducer. In an aspect, BP measurement is continuous.

In an aspect, BP is measured by indirect or non-invasive methods. In an aspect, BP is measured by auscultatory methods. In an aspect, BP is measured by oscillometric methods. In an aspect, BP is measured by palpation. In an aspect, BP is measured by a vascular unloading technique.

In an aspect, BP is measured by tonometry. In an aspect, BP is measured by pulse decomposition analysis. In an aspect, BP is measured by a pulse wave velocity technique.

In an aspect, BP is measured by a sphygmomanometer or a blood pressure gauge. In an aspect, a sphygmomanometer comprises an inflatable cuff, a measuring unit (the mercury manometer, or aneroid gauge), and a mechanism for inflation which may be a manually operated bulb and valve or a pump operated electrically. In an aspect, a sphygmomanometer comprises a manual meter. In an aspect, a sphygmomanometer comprises a digital meter. In an aspect, BP is measured by a digital blood pressure monitor, comprising an inflatable sphygmomanometer cuff and an electronic pressure sensor.

In an aspect, sitting BP is measured when a subject sits in a comfortable chair with the back supported for at least 3 minutes before reading. Both feet of the subject are flat on the ground and both legs are uncrossed. The arm of the subject is rested with the cuff in position but completely deflated, on a table at chest height. In an aspect, systolic BP and diastolic BP are measured using methods known in the art. In an aspect, a cuff of a sphygmomanometer is completely deflated for a full minute between measurements. In an aspect, the size of a cuff is selected and fitted for the patient. In an aspect, measurements are taken from both arms to determine if the pressure is significantly higher in one arm than the other, and if so, the higher reading arm would be used for readings. In an aspect, a BP measurement is the average of two measurements taken with the cuff remaining in position but completely deflated for a full minute between measurements.

In an aspect, the presence or extent of hypertension may be evaluated using other methods known in the art, including, without limitation, general clinical examination to evaluate BP, heart rate, heart rhythm, arterial oxygen, and hemoglobin levels; echocardiography to measure ejection fraction, left ventricle (LV) and left atrial (LA) diameter, LV wall motion, LV filling pressure, and diastolic function by pulse and tissue Doppler; use of a Swan-Ganz catheter to measure cardiac output, pulmonary wedge capillary pressure, pulmonary arterial pressure, right ventricle pressure, right atrial pressure, and systemic and pulmonary vascular resistance; assessment of kidney function by determination of glomerular filtration rate, serum creatinine, and blood urea nitrogen; and measurement of biomarkers such as B-type natriuretic peptide (BNP), amino-terminal proBNP (NT-proBNP), troponin-T, troponin-I, C-reactive protein (CRP), and creatine kinase, serum cystatin-C, albuminuria, neutrophil gelatinase associated lipocalin (NGAL), Nacetyl-beta-D-glucosaminidase (NAG), kidney injury molecule-1 (KIM-1), angiotensin-II, renin, aldosterone, and inflammatory cytokines (e.g., interleukin (IL)-6, IL-18, etc.).

### Hypertension

As used herein, “hypertension” refers to a medical condition where blood pressure in the arteries is elevated. In an aspect, hypertension exists when the systolic BP is at least 140 mmHg while sitting. In an aspect, hypertension exists when the diastolic BP is at least 90 mmHg while sitting. In an aspect, hypertension in a diabetic patient exists when the systolic BP is at least 130 mmHg while sitting. In an aspect, hypertension in a diabetic patient exists when the diastolic BP is at least 80 mmHg while sitting. In an aspect, hypertension exists when the systolic BP is at least 140 mmHg and the diastolic BP is at least 90 mmHg of a diastolic BP. In an aspect, hypertension in a diabetic patient exists when the systolic BP is at least 130 mmHg and the diastolic BP is at least 80 mmHg of a diastolic BP. In an aspect, a patient in need of treatment for hypertension has a systolic blood pressure of greater than or equal to about 140 mmHg prior to the administering. In an aspect, a patient in need of lowering systolic blood pressure has a systolic blood pressure of greater than or equal to about 140 mmHg prior to the administering.

As used herein, “resistant hypertension” (RH) of “difficult-to-control hypertension” (DTC) refers to blood pressure that remains above 140 mmHg systolic BP and 90 mmHg diastolic BP, in spite of a patient being prescribed three or more antihypertensive drugs simultaneously with different mechanisms of action, including a diuretic. Other antihypertensive drugs that work through different mechanisms of action, include but are not limited to Ca²⁺ channel blockers, ACE inhibitors, angiotensin II receptor blockers (ARB), alpha-blockers, and beta-blockers.

Without being bound by theory, diuretics help the kidneys remove salt and water through urine, lowering the amount of fluid flowing through the veins and arteries, thereby reducing blood pressure. In an aspect, a method for lowering blood pressure in a patient in need thereof further comprises administering a diuretic. In an aspect, a diuretic is chlorothiazide, chlorthalidone, hydrochlorothiazide, indapamide, metolazone, bumetanide, ethacrynic acid, furosemide, torsemide, amiloride, eplerenone, spironolactone, or triamterene. In an aspect, a diuretic is selected from the group consisting of hydrochlorothiazide, spironolactone, bumetanide, chlorthalidone, furosemide, and hydralazine.

Without being bound by theory, Ca²⁺ channel blockers block calcium from entering the cells of the heart and arteries, allowing blood vessels to relax and open, thereby lowering blood pressure. Some Ca²⁺ channel blockers may also slow the heart rate, further lowing blood pressure. In an aspect, the method for lowering blood pressure in a patient in need thereof further comprises administering a Ca²⁺ channel blocker. In an aspect, a Ca²⁺ channel blocker is amlodipine, diltiazem, felodipine, isradipine, nicardipine, nifedipine, nisoldipine, or verapamil. In an aspect, a Ca²⁺ channel blocker is selected from the group consisting of amlodipine besilate, verapamil, diltiazem, and nifedipine.

Without being bound by theory, ACE inhibitors block the production of angiotensin 2 which narrows blood vessels, thereby lowering blood pressure. Angiotensin 2 also releases hormones that raise blood pressure. In an aspect, a method for lowering blood pressure in a patient in need thereof further comprises administering an ACE inhibitor. In an aspect, an ACE inhibitor is benazepril, captopril, enalapril, fosinopril, lisinopril, moexipril, perindopril, quinapril, ramipril, or trandolapril. In an aspect, an ACE inhibitor is selected from the group consisting of benazepril and lisinopril. Without being bound by theory, angiotensin II receptor blockers (ARBs) stop the action of angiotensin 2. In an aspect, the method for lowering blood pressure in a patient in need thereof further comprises administering an ARB. In an aspect, the ARB is azilsartan, candesartan, irbesartan, losartan, Olmesartan, telmisartan, or valsartan. In an aspect, the ARB is selected from the group consisting of losartan and olmesartan.

Without being bound by theory, alpha-blockers lower blood pressure by keeping a hormone called norepinephrine from tightening the muscles in the walls of smaller arteries and veins. As a result, the blood vessels remain open and relaxed. This improves blood flow and lowers blood pressure. In an aspect, a method for lowering blood pressure in a patient in need thereof further comprises administering an alpha-blocker. In an aspect, an alpha-blocker is doxazosin, prazosin, tamsulosin, clonidine, or terazosin. In an aspect, an alpha-blocker is selected from the group consisting of doxazosin, prazosin, tamsulosin, and clonidine.

Without being bound by theory, beta-blockers block the effects of the hormone epinephrine, also known as adrenaline, and cause the heart to beat more slowly with less force, thereby lowering blood pressure. In an aspect, a method for lowering blood pressure in a patient in need thereof further comprises administering a beta-blocker. In an aspect, a beta-blocker is acebutolol, atenolol, bisoprolol, carvedilol, metoprolol, nadolol, nebivolol, or propanolol. In an aspect, a beta-blocker is selected from the group consisting of carvedilol, atenolol, bisoprolol, metoprolol, and nebivolol.

In an aspect, a patient in need of treatment for resistant hypertension is prescribed three antihypertensive drugs that includes a diuretic prior to the treatment using the methods disclosed herein. In an aspect, a patient in need of treatment for resistant hypertension is prescribed four antihypertensive drugs that includes a diuretic prior to the treatment using the methods disclosed herein. In an aspect, a patient in need of treatment for resistant hypertension is prescribed five antihypertensive drugs that includes a diuretic prior to the treatment using the methods disclosed herein. In an aspect, a patient in need of treatment for resistant hypertension is prescribed three antihypertensive drugs that includes an ACE inhibitor prior to the treatment using the methods disclosed herein. In an aspect, a patient in need of treatment for resistant hypertension is prescribed four antihypertensive drugs that includes an ACE inhibitor prior to the treatment using the methods disclosed herein. In an aspect, a patient in need of treatment for resistant hypertension is prescribed five antihypertensive drugs that includes an ACE inhibitor prior to the treatment using the methods disclosed herein. In an aspect, a patient in need of treatment for resistant hypertension is prescribed three antihypertensive drugs that includes an ARB prior to the treatment using the methods disclosed herein. In an aspect, a patient in need of treatment for resistant hypertension is prescribed four antihypertensive drugs that includes an ARB inhibitor prior to the treatment using the methods disclosed herein. In an aspect, a patient in need of treatment for resistant hypertension is prescribed five antihypertensive drugs that includes an ACE inhibitor prior to the treatment using the methods disclosed herein. In an aspect, a patient in need of treatment for resistant hypertension is taking 3 or more anti-hypertensive medications prior to the treatment using the methods disclosed herein.

In an aspect, a patient in need of lowering blood pressure is prescribed three antihypertensive drugs that includes a diuretic prior to the treatment using the methods disclosed herein. In an aspect, a patient in need of lowering blood pressure is prescribed four antihypertensive drugs that includes a diuretic prior to the treatment using the methods disclosed herein. In an aspect, a patient in need of lowering blood pressure is prescribed five antihypertensive drugs that includes a diuretic prior to the treatment using the methods disclosed herein. In an aspect, a patient in need of lowering blood pressure is prescribed three antihypertensive drugs that includes an ACE inhibitor prior to the treatment using the methods disclosed herein. In an aspect, a patient in need of lowering blood pressure is prescribed four antihypertensive drugs that includes an ACE inhibitor prior to the treatment using the methods disclosed herein. In an aspect, a patient in need of lowering blood pressure is prescribed five antihypertensive drugs that includes an ACE inhibitor prior to the treatment using the methods disclosed herein. In an aspect, a patient in need of lowering blood pressure is prescribed three antihypertensive drugs that includes an ARB prior to the treatment using the methods disclosed herein. In an aspect, a patient with in need of lowering blood pressure is prescribed four antihypertensive drugs that includes an ARB inhibitor prior to the treatment using the methods disclosed herein. In an aspect, a patient in need of lowering blood pressure is prescribed five antihypertensive drugs that includes an ACE inhibitor prior to the treatment using the methods disclosed herein. In an aspect, a patient in need of in need of lowering blood pressure is taking 3 or more anti-hypertensive medications prior to the treatment using the methods disclosed herein. In an aspect, a patient in need of lowering systolic blood pressure is taking 3 or more anti-hypertensive medications prior to the treatment using the methods disclosed herein.

In an aspect, the methods disclosed herein further comprises administering one or more anti-hypertensive medications. In an aspect, a patient in need of in need of treatment for resistant hypertension by the methods disclosed herein, are further administered one or more anti-hypertensive medications. In an aspect, a patient in need of in need of lowering blood pressure by the methods disclosed herein, are further administered one or more anti-hypertensive medications.

As used herein, “obese” or “obesity” refers to a body mass index (BMI) greater than 29.9 kg/m². In an aspect, a patient identified as obese has a BMI greater than or equal to 30 kg/m². In an aspect, a patient diagnosed as obese has a BMI of 30-40 kg/m². In an aspect, BMI is calculated by dividing an individual's weight in pounds (lb) by the square of the height in inches (in) and multiplying by a conversion factor of 703, rounded to one decimal place. In an aspect, BMI is calculated by dividing an individual's weight in kilograms (kg) by the square of the height in meters (m), rounded to one decimal place.

In an aspect, a patient in need of lowering blood pressure has hypertension. In an aspect, a patient in need of lowering blood pressure has resistant hypertension. In an aspect, a patient in need of lowering blood pressure has difficult-to-control hypertension. In an aspect, a patient in need of treatment for resistant hypertension is at least 18 years old. In an aspect, a patient in need of lowering blood pressure is 18-80 years old. In an aspect, a patient in need of treatment for resistant hypertension is at least 18 years old. In an aspect, a patient in need of lowering blood pressure is 18-80 years old.

In an aspect, a patient in need of treatment for resistant hypertension has a body mass index (BMI) of at least 18 kg/m² prior to the administering. In an aspect, a patient in need of treatment for resistant hypertension has a BMI of less than 40 kg/m² prior to the administering. In an aspect, a patient in need of treatment for resistant hypertension has a BMI of 18-40 kg/m² prior to the administering. In an aspect, a patient in need of treatment for resistant hypertension is obese prior to the administering. In an aspect, a patient in need of treatment for resistant hypertension has a BMI greater than or equal to 29.9 kg/m² prior to the administering.

In an aspect, a patient in need of lowering blood pressure has a body mass index (BMI) of at least 18 kg/m². In an aspect, a patient in need of lowering blood pressure has a BMI of less than 40 kg/m². In an aspect, a patient in need of lowering blood pressure has a BMI of 18-40 kg/m². In an aspect, a patient in need of lowering blood pressure is obese. In an aspect, a patient in need of lowering blood pressure has a BMI greater than or equal to 29.9 kg/m².

In an aspect, a patient's estimated glomerular filtration rate (eGFR) is a measure of the patient's kidney function. Without being bound by theory, creatinine is a breakdown product of creatine phosphate from muscle and protein metabolism, that is released at a constant rate by the body into the blood. Creatinine is removed from blood by the kidneys through glomerular filtration, thus blood levels of creatinine may be used to calculate eGFR. In an aspect, eGFR is calculated using the Modification of Diet in Renal Disease Study (MDRD) equation. See e.g., Levey A S, Bosch J P, Lewis J B, Greene T, Rogers N, Roth D, “A more accurate method to estimate glomerular filtration rate from serum creatinine: a new prediction equation,” Modification of Diet in Renal Disease Study Group, Ann. Intern. Med., 130:461-70 (1999). In an aspect, eGFR is calculated using the 4-variable Modification of Diet in Renal Disease Study equation (MDRD-4). See e.g., Levey A S, Coresh J, Greene T, et al., “Using standardized serum creatinine values in the modification of diet in renal disease study equation for estimating glomerular filtration rate,” Ann. Intern. Med., 145:247-54 (2006). In an aspect, eGFR is calculated using the CKD Epidemiology Collaboration (CKD-EPI) method. See e.g., Levey A S, et al., “A new equation to estimate glomerular filtration rate,” Ann Intern Med., 150 (9): 604-12 (2009); Delgado et al., “A Unifying Approach for GFR Estimation: Recommendations of the NKF-ASN Task Force on Reassessing the Inclusion of Race in Diagnosing Kidney Disease,” Am. J. Kidney Diseases, 79 (2): 268-288 (2022).

In an aspect, a patient in need of lowering blood pressure has a CKD-EPI eGFR of at least 30 mL/min/1.73 m². In an aspect, a patient in need of lowering blood pressure has a CKD-EPI eGFR of equal to or greater than 90 mL/min/1.73 m². In an aspect, a patient in need of lowering blood pressure has a CKD-EPI eGFR of about 68-89 mL/min/1.73 m². In an aspect, a patient in need of lowering blood pressure has a CKD-EPI eGFR of about 45-59 mL/min/1.73 m². In an aspect, a patient in need of lowering blood pressure has a CKD-EPI eGFR of about 30-44 mL/min/1.73 m².

In an aspect, a patient in need of treatment for resistant hypertension has a CKD-EPI eGFR of between 20-29 mL/min/1.73 m² prior to the administering. In an aspect, a patient in need of lowering blood pressure has a CKD-EPI eGFR of between 20-29 mL/min/1.73 m² prior to the administering. In an aspect, a patient in need of treatment for resistant hypertension has a CKD-EPI eGFR of greater than or equal to 30 mL/min/1.73 m prior to the administering. In an aspect, a patient in need of lowering blood pressure has a CKD-EPI eGFR of greater than or equal to 30 mL/min/1.73 m prior to the administering.

### Other Indications

In an aspect, a patient in need of lowering blood pressure has a hypertensive crisis. As used herein, a “hypertensive crisis” refers to an acute, marked elevation in blood pressure. A hypertensive crisis includes hypertensive emergency (severe elevation in BP (>180/120 mmHg) complicated by impending or progressive target-organ dysfunction) and hypertensive urgency (severe elevation in BP without signs of organ damage). In an aspect, a hypertensive crisis may be perioperative hypertension. In an aspect, a hypertensive crisis may be postoperative hypertension. In an aspect, a hypertensive crisis may be acute severe hypertension. In an aspect, a hypertensive crisis may occur during an intracerebral hemorrhage. In an aspect, a hypertensive crisis is a hypertensive emergency. In an aspect, a patient in need of lowering systolic blood pressure has a condition selected from the group consisting of perioperative hypertension, postoperative hypertension, acute severe hypertension, intracerebral hemorrhage, hypertensive emergency, and hypertensive crisis.

### MANP

As used herein, an “MANP” is an ANP-based peptide having an amino acid sequence that includes the 28 amino acid mature human ANP sequence with an additional 12 amino acid carboxy terminus. Without being bound by theory, MANP is a pGC-A/cGMP activator that can significantly lower blood pressure and vascular resistance. International Patent Application Nos. PCT/US2017/060808 and PCT/US2023/074449 disclose analogues of MANP that can be used to treat cardiorenal and metabolic disease and are incorporated herein in their entireties.

In an aspect, MANP comprises a peptide with the sequence set forth in SEQ ID NO:1 SLRRSSCFGGRMDRIGAQSGLGCNSFRYRITAREDKQGWA. In an aspect, an MANP can be a variant of the sequence set forth in SEQ ID NO: 1. In an aspect, an MANP can contain the amino acid sequence set forth in SEQ ID NO:1, together with one or more amino acid additions, subtractions, or substitutions. In an aspect, an MANP can contain the amino acid sequence set forth in SEQ ID NO:1, together with one or more amino acid additions, subtractions, and substitutions. In an aspect, an MANP can contain the amino acid sequence set forth in SEQ ID NO:1, together with one, two, three, four, five, six, seven, eight, nine, or ten single amino acid residue additions, subtractions, or substitutions. In an aspect, an MANP can contain the amino acid sequence set forth in SEQ ID NO:1, together with one, two, three, four, five, six, seven, eight, nine, or ten single amino acid residue additions, subtractions, and substitutions.

In an aspect, any amino acid residue set forth in SEQ ID NO:1 can be subtracted, and any amino acid residue (e.g., any of the 20 conventional amino acid residues or any other type of amino acid such as ornithine or citrulline) can be added to the sequence set forth in SEQ ID NO:1. In an aspect, an MANP can contain one or more chemical structures such as e-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; and/or glycosylated amino acids such as amino acids containing monosaccharides (e.g., D-glucose, D-galactose, D-mannose, D-glucosamine, and D-galactosamine) or combinations of monosaccharides.

MANPs having one or more amino acid additions, subtractions, or substitutions relative to the representative MANP sequence set forth in SEQ ID NO:1, also referred to herein as “variant” MANPs, can be generated using any suitable method. In an aspect, amino acid substitutions can be made by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful conservative substitutions can include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine.

In an aspect, an MANP can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the polypeptide variant using, for example, methods disclosed herein.

In an aspect, an MANP can have a length of, for example, 35 to 45 amino acid residues (e.g., 35 to 40, 40 to 45, 35 to 37, 36 to 38, 37 to 39, 38 to 40, 39 to 41, 40 to 42, 41 to 43, 42 to 44, or 43 to 45 amino acid residues).

In an aspect, an MANP may include an amino acid sequence with at least 90% (e.g., at least 90%, at least 92.5%, at least 95%, at least 97.5%, or 100%) sequence identity to the sequence set forth in SEQ ID NO:1. Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences.

In an aspect, a percent sequence identity between a particular nucleic acid or amino acid sequence and a sequence referenced by a particular sequence identification number may be determined as follows. First, a nucleic acid or amino acid sequence is compared to the sequence set forth in a particular sequence identification number using the BLAST 2 Sequences (Bl2seq) program from 25 the stand-alone version of BLASTZ containing BLASTN version 2.0.14 and BLASTP version 2.0.14. This stand-alone version of BLASTZ may be obtained online at fr.com/blast or at ncbi.nlm.nih.gov. Instructions explaining how to use the Bl2seq program may be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. In an aspect, the following command may be used to generate an output file containing a comparison between two sequences: C:\Bl2seq-i c:\seq1.txt -j c:\seq2.txt-p blastn-o c:\output.txt -q -1 -r 2. To compare two amino acid sequences, the options of Bl2seq are set as follows:-i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. In an aspect, the following command may be used to generate an output file containing a comparison between two amino acid sequences: C:\Bl2seq -I c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 20 consecutive nucleotides or amino acid residues from a sequence set forth in an identified sequence), followed by multiplying the resulting value by 100. In an aspect, an amino acid sequence that has 37 matches when aligned with the sequence set forth in SEQ ID NO:1 is 25 92.5 percent identical to the sequence set forth in SEQ ID NO:1 (i.e., 37-40×100=92.5). It is noted that the percent sequence identity value is rounded to the nearest tenth. In an aspect, 75.11, 75.12, 75.13, and 75.14 are rounded down to 75.1, while 75.15, 75.16, 75.17, 75.18, and 75.19 are rounded up to 75.2. It also is noted that the length value will always be an integer.

In an aspect, this disclosure provides methods of treating resistant hypertension in a patient in need thereof, comprising administering to the patient fixed doses of MANP, wherein the MANP is a peptide having an amino acid sequence consisting essentially of SEQ ID NO:1. In an aspect, this disclosure provides methods of lowering blood pressure in a patient in need thereof, comprising administering to the patient fixed doses of MANP, wherein the MANP is a peptide having an amino acid sequence consisting essentially of SEQ ID NO:1.

### Fixed Dosage

This disclosure is based in part on the surprising result that fixed doses of MANP is effective in lowering blood pressure in patients in need thereof, or in patients in need of treatment for resistant hypertension.

In an aspect, a method for treating resistant hypertension comprises administering one or more doses of MANP at a dose effective for lowering blood pressure. In an aspect, a dose of MANP is at least 200 mcg. In an aspect, a dose of MANP is at least 220 mcg. In an aspect, a dose of MANP is at least 240 mcg. In an aspect, a dose of MANP is at least 260 mcg. In an aspect, a dose of MANP is at least 280 mcg. In an aspect, a dose of MANP is at least 300 mcg. In an aspect, a dose of MANP is at least 350 mcg. In an aspect, a dose of MANP is at least 400 mcg. In an aspect, a dose of MANP is at least 500 mcg. In an aspect, a dose of MANP is at least 550 mcg. In an aspect, a dose of MANP is at least 600 mcg. In an aspect, a dose of MANP is about 200 mcg. In an aspect, a dose of MANP is about 220 mcg. In an aspect, a dose of MANP is about 240 mcg. In an aspect, a dose of MANP is about 260 mcg. In an aspect, a dose of MANP is about 280 mcg. In an aspect, a dose of MANP is about 300 mcg. In an aspect, a dose of MANP is about 350 mcg. In an aspect, a dose of MANP is about 400 mcg. In an aspect, a dose of MANP is about 500 mcg. In an aspect, a dose of MANP is about 550 mcg. In an aspect, a dose of MANP is about 600 mcg. In an aspect, a dose of MANP is about 100 mcg to about 800 mcg. In an aspect, a dose of MANP is about 200 mcg to about 700 mcg. In an aspect, a dose of MANP is about 300 mcg to about 600 mcg. In an aspect, a dose of MANP is about 250 mcg to about 350 mcg. In an aspect, a dose of MANP is about 280 mcg to about 320 mcg. In an aspect, a dose of MANP is about 300 mcg. In an aspect, a dose of MANP is about 400 mcg to about 500 mcg. In an aspect, a dose of MANP is about 430 mcg to about 470 mcg. In an aspect, a dose of MANP is about 450 mcg. In an aspect, a dose of MANP is about 550 mcg to about 650 mcg. In an aspect, a dose of MANP is about 580 mcg to about 620 mcg. In an aspect, a dose of MANP is about 600 mcg.

In an aspect, a method for lowering blood pressure comprises administering one or more doses of MANP at a dose effective for lowering blood pressure. In an aspect, a dose of MANP is at least 200 mcg. In an aspect, a dose of MANP is at least 220 mcg. In an aspect, a dose of MANP is at least 240 mcg. In an aspect, a dose of MANP is at least 260 mcg. In an aspect, a dose of MANP is at least 280 mcg. In an aspect, a dose of MANP is at least 300 mcg. In an aspect, a dose of MANP is at least 350 mcg. In an aspect, a dose of MANP is at least 400 mcg. In an aspect, a dose of MANP is at least 500 mcg. In an aspect, a dose of MANP is at least 550 mcg. In an aspect, a dose of MANP is at least 600 mcg. In an aspect, a dose of MANP is about 200 mcg. In an aspect, a dose of MANP is about 220 mcg. In an aspect, a dose of MANP is about 240 mcg. In an aspect, a dose of MANP is about 260 mcg. In an aspect, a dose of MANP is about 280 mcg. In an aspect, a dose of MANP is about 300 mcg. In an aspect, a dose of MANP is about 350 mcg. In an aspect, a dose of MANP is about 400 mcg. In an aspect, a dose of MANP is about 500 mcg. In an aspect, a dose of MANP is about 550 mcg. In an aspect, a dose of MANP is about 600 mcg. In an aspect, a dose of MANP is about 100 mcg to about 800 mcg. In an aspect, a dose of MANP is about 200 mcg to about 700 mcg. In an aspect, a dose of MANP is about 300 mcg to about 600 mcg. In an aspect, a dose of MANP is about 250 mcg to about 350 mcg. In an aspect, a dose of MANP is about 280 mcg to about 320 mcg. In an aspect, a dose of MANP is about 300 mcg. In an aspect, a dose of MANP is about 400 mcg to about 500 mcg. In an aspect, a dose of MANP is about 430 mcg to about 470 mcg. In an aspect, a dose of MANP is about 450 mcg. In an aspect, a dose of MANP is about 550 mcg to about 650 mcg. In an aspect, a dose of MANP is about 580 mcg to about 620 mcg. In an aspect, a dose of MANP is about 600 mcg.

In an aspect, a method disclosed herein comprise administering one or more doses of MANP. In an aspect, a method disclosed herein comprise administering more than one dose of MANP. In an aspect, a method disclosed herein comprise administering at least two doses, at least three doses, at least four doses, at least five doses, at least six doses, at least seven doses, at least eight doses, at least nine doses, at least ten doses, at least 11 doses, at least 12 doses, at least 13 doses, at least 14 doses, at least 15 doses, at least 16 doses, at least 17 doses, at least 18 doses, at least 19 doses, at least 20 doses, at least 30 doses, at least 40 doses, at least 50 doses, at least 60 doses, at least 70 doses, at least 80 doses, at least 90 doses, at least 100 doses of MANP.

In an aspect, a method disclosed herein comprise administering one or more doses of MANP at fixed time intervals. In an aspect, doses are administered at least once every 6 hours, at least once every 12 hours, at least once every 18 hours, at least once every 24 hours, at least once every 48 hours, at least once every 72 hours, at least once every week, at least once every two weeks, or at least once a month. In an aspect, the doses are administered once every 24 hours.

### BP Monitoring

In an aspect, an initial blood pressure in a patient in need thereof is measured before receiving any dose of MANP. In an aspect, a blood pressure is monitored at a single timepoint. In an aspect, at least one, at least two, at least three, at least four, at least five, at least six, at least seven, at least eight, at least nine, or at least ten BP measurements is taken before receiving any doses of MANP. In an aspect, an initial blood pressure in a patient in need thereof is measured at least 5 min, at least 10 min, at least 15 min, at least 20 min, at least 30 min, at least 35 min, at least 40 min, at least 45 min, at least 50 min, at least 55 min, at least 60 min, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 2 days, at least 3 days, at least 4 days, at least 5 days, at least 6 days, at least 7 days, at least 8 days, at least 9 days, at least 10 days, at least 2 weeks, at least 3 weeks, or at least 4 weeks before receiving any dose of MANP. In an aspect, an initial blood pressure in a patient in need thereof is measured about 5 min, about 10 min, about 15 min, about 20 min, about 30 min, about 35 min, about 40 min, about 45 min, about 50 min, about 55 min, about 60 min, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 2 days, about 3 days, about 4 days, about 5 days, about 6 days, about 7 days, about 8 days, about 9 days, about 10 days, about 2 weeks, about 3 weeks, or about 4 weeks before receiving any dose of MANP.

In an aspect, a first blood pressure of a patient in need thereof is measured at a first timepoint after a dose of MANP. In an aspect, a first timepoint is about 5 min, about 10 min, about 15 min, about 20 min, about 30 min, about 35 min, about 40 min, about 45 min, about 50 min, about 55 min, about 60 min, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours after receiving a dose of MANP. In an aspect, a first timepoint is at least 5 min, at least 10 min, at least 15 min, at least 20 min, at least 30 min, at least 35 min, at least 40 min, at least 45 min, at least 50 min, at least 55 min, at least 60 min, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, or at least 24 hours after receiving a dose of MANP.

In an aspect, blood pressure is monitored continuously. As used herein, “ambulatory blood pressure (ABP)” is the blood pressure over the course of the full 24-hour sleep-wake cycle. Without being bound by theory, ABP monitoring at regular intervals throughout the day and night avoids white coat hypertension, detects masked hypertension, and provides a more nuanced reading that can take into account physiological variations of blood pressure throughout the day. In an aspect, the ABP is measured. In aspect, an ABP is measured before receiving any dose of MANP. In aspect, an ABP is measured after receiving a dose of MANP. In aspect, a single timepoint blood pressure is measured before receiving any dose of MANP and an ABP is measured after receiving a dose of MANP. In aspect, an ABP is measured before receiving any dose of MANP and an ABP is measured after receiving a dose of MANP. In aspect, an ABP is measured before receiving any dose of MANP and a single timepoint blood pressure is measured after receiving a dose of MANP. In aspect, a single timepoint blood pressure is measured before receiving any dose of MANP and a single timepoint blood pressure is measured after receiving a dose of MANP.

In an aspect, blood pressure in a patient in need thereof is measured at a first timepoint after one of the one or more doses of MANP is reduced compared to an initial blood pressure measured before receiving the first dose of MANP. In an aspect, an ABP in a patient in need thereof measured after one of the one or more doses of MANP is reduced compared to an initial blood pressure measured before receiving the first dose of MANP. In an aspect, a systolic or diastolic blood pressure in a patient in need thereof measured at a first timepoint after one of the one or more doses of MANP is reduced by at least 1 mmHg, at least 2 mmHg, at least 3 mmHg, at least 4 mmHg, at least 5 mm Hg, at least 6 mmHg, at least 7 mmHg, at least 8 mmHg, at least 9 mmHg, at least 10 mmHg, at least 11 mmHg, at least 12 mmHg, at least 13 mmHg, at least 14 mmHg, at least 15 mmHg, at least 16 mmHg, at least 17 mmHg, at least 18 mmHg, at least 19 mmHg, at least 20 mmHg, at least 25 mmHg, at least 30 mmHg, at least 35 mmHg, at least 40 mmHg, at least 45 mmHg, at least 50 mmHg, at least 55 mmHg, at least 60 mmHg, at least 65 mmHg, at least 70 mmHg, at least 75 mmHg, at least 80 mmHg, at least 85 mmHg, at least 90 mmHg, at least 95 mmHg, at least 100 mmHg, at least 105 mmHg, at least 110 mmHg, at least 115 mmHg, or at least 120 mmHg. In an aspect, a systolic or diastolic blood pressure in a patient in need thereof measured at a first timepoint after one of the one or more doses of MANP is reduced by about 1 mmHg, about 2 mmHg, about 3 mmHg, about 4 mmHg, about 5 mm Hg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 25 mmHg, about 30 mmHg, about 35 mmHg, about 40 mmHg, about 45 mmHg, about 50 mmHg, about 55 mmHg, about 60 mmHg, about 65 mmHg, about 70 mmHg, about 75 mmHg, about 80 mmHg, about 85 mmHg, about 90 mmHg, about 95 mmHg, about 100 mmHg, about 105 mmHg, about 110 mmHg, about 115 mmHg, or about 120 mmHg.

In an aspect, a systolic or diastolic blood pressure in a patient in need thereof measured at a first timepoint after one of the one or more doses of MANP is reduced by at least 2%, at least 4%, at least 6%, at least 8%, at least 10%, at least 12%, at least 14%, at least 16%, at least 18%, at least 20%, at least 22%, at least 24%, at least 26%, at least 28%, at least 30%, at least 32%, at least 34%, at least 36%, at least 38%, at least 40%, at least 42%, at least 44%, at least 46%, at least 48%, or at least 50%.

In an aspect, a patient in need of treatment with a method disclosed herein has a systolic blood pressure of no more than 120 mmHg, no more than 122 mmHg, no more than 124 mmHg, no more than 126 mmHg, no more than 128 mmHg, no more than 130 mmHg, no more than 132 mmHg, no more than 134 mmHg, no more than 136 mmHg, no more than 138 mmHg, no more than 140 mmHg, no more than 142 mmHg, no more than 144 mmHg, no more than 146 mmHg, no more than 148 mmHg, no more than 150 mmHg, no more than 152 mmHg, no more than 154 mmHg, no more than 156 mmHg, no more than 158 mmHg, no more than 160 mmHg, no more than 162 mmHg, no more than 164 mmHg, no more than 166 mmHg, no more than 168 mmHg, no more than 170 mmHg, no more than 172 mmHg, no more than 174 mmHg, no more than 176 mmHg, no more than 178 mmHg, no more than 180 mmHg, no more than 182 mmHg, no more than 184 mmHg, no more than 186 mmHg, no more than 188 mmHg, no more than 190 mmHg, no more than 192 mmHg, no more than 194 mmHg, no more than 196 mmHg, no more than 198 mmHg, or no more than 200 mmHg after the administering.

As used herein, the term “hypotension” refers to a low blood pressure, with a systolic blood pressure of less than 90 mmHg or a diastolic blood pressure of less than 60 mmHg. As used herein, the term “hypotensive crisis” or “hypotensive shock” refers to a severe or sudden drop in blood pressure, with a systolic blood pressure of less than 90 mmHg or a diastolic blood pressure of less than 60 mmHg, that can lead to organ damage or death. A hypotensive crisis can arise from a variety of physiological conditions, including but not limited to, perioperative complications, blood loss (shock), heart attacks, severe infections, anaphylaxis (allergic reaction), medications, and spinal anesthesia. Methods disclosed herein lower blood pressure of patients in need thereof, without triggering a hypotensive crisis. In an aspect, a patient in need of treatment with the methods disclosed herein has a systolic blood pressure of at least 60 mmHg, at least 62 mmHg, at least 64 mmHg, at least 66 mmHg, at least 68 mmHg, at least 70 mmHg, at least 72 mmHg, at least 74 mmHg, at least 76 mmHg, at least 78 mmHg, at least 80 mmHg, at least 82 mmHg, at least 84 mmHg, at least 86 mmHg, at least 88 mmHg, at least 90 mmHg, at least 92 mmHg, at least 94 mmHg, at least 96 mmHg, at least 98 mmHg, or at least 100 mmHg after the administering, without triggering a hypotensive crisis. In an aspect, a patient in need of treatment with the methods disclosed herein has a systolic blood pressure of at least 80 mmHg after the administering. In an aspect, the methods disclosed herein lower blood pressure by about 2 mmHg, about 4 mmHg, about 6 mmHg, about 8 mmHg, about 10 mmHg, about 12 mmHg, about 14 mmHg, about 16 mmHg, about 18 mmHg, about 20 mmHg, about 22 mmHg, about 24 mmHg, about 26 mmHg, about 28 mmHg, about 30 mmHg, about 32 mmHg, about 34 mmHg, about 36 mmHg, about 38 mmHg, about 40 mmHg, about 42 mmHg, about 44 mmHg, about 46 mmHg, about 48 mmHg, about 50 mmHg, about 52 mmHg, about 54 mmHg, about 56 mmHg, about 58 mmHg, about 60 mmHg, about 62 mmHg, about 64 mmHg, about 66 mmHg, about 68 mmHg, about 70 mmHg, about 72 mmHg, about 74 mmHg, about 76 mmHg, about 78 mmHg, about 80 mmHg, about 82 mmHg, about 84 mmHg, about 86 mmHg, about 88 mmHg, about 90 mmHg, about 92 mmHg, about 94 mmHg, about 96 mmHg, about 98 mmHg, or about 100 mmHg after the administering, without triggering a hypotensive crisis. In an aspect, the methods disclosed herein lower blood pressure by about 2-80 mmHg, about 4-60 mmHg, about 10-40 mmHg, about 10-30 mmHg, about 20-50 mmHg after the administering, without triggering a hypotensive crisis.

cGMP Monitoring

In an aspect, an initial plasma cGMP is measured or calculated in a patient in need thereof, before receiving any dose of MANP. In an aspect, at least one, at least two, at least three, at least four, at least five, at least six, at least seven, at least eight, at least nine, or at least ten plasma cGMP measurements are taken before receiving any doses of MANP. In an aspect, an initial plasma cGMP measurement is taken at least 5 min, at least 10 min, at least 15 min, at least 20 min, at least 30 min, at least 35 min, at least 40 min, at least 45 min, at least 50 min, at least 55 min, at least 60 min, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 2 days, at least 3 days, at least 4 days, at least 5 days, at least 6 days, at least 7 days, at least 8 days, at least 9 days, at least 10 days, at least 2 weeks, at least 3 weeks, or at least 4 weeks before receiving any dose of MANP. In an aspect, an initial plasma cGMP measurement is taken about 5 min, about 10 min, about 15 min, about 20 min, about 30 min, about 35 min, about 40 min, about 45 min, about 50 min, about 55 min, about 60 min, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 2 days, about 3 days, about 4 days, about 5 days, about 6 days, about 7 days, about 8 days, about 9 days, about 10 days, about 2 weeks, about 3 weeks, or about 4 weeks before receiving any dose of MANP.

In an aspect, a first plasma cGMP measurement is taken in a patient in need thereof, at a first timepoint after a dose of MANP. In an aspect, a first plasma cGMP measurement is taken at 5 min, about 10 min, about 15 min, about 20 min, about 30 min, about 35 min, about 40 min, about 45 min, about 50 min, about 55 min, about 60 min, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 2 days, or about 3 days after receiving a dose of MANP. In an aspect, a first timepoint is at least 5 min, at least 10 min, at least 15 min, at least 20 min, at least 30 min, at least 35 min, at least 40 min, at least 45 min, at least 50 min, at least 55 min, at least 60 min, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 2 days, or at least 3 days after receiving a dose of MANP.

In an aspect, a plasma cGMP measured in a patient in need thereof at a first timepoint after one of the one or more doses of MANP is administered, is compared to an initial plasma cGMP measured before receiving the first dose of MANP. In an aspect, a plasma cGMP measured in a patient in need thereof at a first timepoint after one of the one or more doses of MANP is administered, is equal to or greater than an initial plasma cGMP measured before receiving the first dose of MANP. In an aspect, a plasma cGMP measured in a patient in need thereof at a first timepoint after one of the one or more doses of MANP is administered, is increased by at least 1 μmol/mL, at least 2 μmol/mL, at least 3 μmol/mL, at least 4 μmol/mL, at least 5 μmol/mL, at least 6 μmol/mL, at least 7 μmol/mL, at least 8 μmol/mL, at least 9 μmol/mL, at least 10 μmol/mL, at least 11 μmol/mL, at least 12 μmol/mL, at least 13 μmol/mL, at least 14 μmol/mL, at least 15 μmol/mL, at least 16 pmol/mL, at least 17 μmol/mL, at least 18 μmol/mL, at least 19 μmol/mL, at least 20 μmol/mL, at least 25 μmol/mL, at least 30 μmol/mL, at least 35 μmol/mL, at least 40 μmol/mL, at least 45 pmol/mL, or at least 50 μmol/mL compared to that measured before receiving the first dose of MANP. In an aspect, a patient's plasma cGMP measured at a first timepoint after one of the one or more doses of MANP is increased by about 1 μmol/mL, about 2 μmol/mL, about 3 μmol/mL, about 4 μmol/mL, about 5 μmol/mL, about 6 μmol/mL, about 7 μmol/mL, about 8 μmol/mL, about 9 pmol/mL, about 10 μmol/mL, about 11 μmol/mL, about 12 μmol/mL, about 13 μmol/mL, about 14 pmol/mL, about 15 μmol/mL, about 16 μmol/mL, about 17 μmol/mL, about 18 μmol/mL, about 19 pmol/mL, about 20 μmol/mL, about 25 μmol/mL, about 30 μmol/mL, about 35 μmol/mL, about 40 pmol/mL, about 45 μmol/mL, or about 50 μmol/mL compared to that measured before receiving the first dose of MANP.

In an aspect, a plasma cGMP measured in a patient in need thereof, at a first timepoint after one of the one or more doses of MANP is administered is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000% compared to that measured before receiving the first dose of MANP.

### EXAMPLES

### Example I—a Phase 1b, Multiple Ascending Dose Trial Examining the Effect of Subcutaneous MANP on Blood Pressure in Difficult to Control/Resistant Hypertensive Subjects

The effect of MANP on blood pressure is evaluated at ascending dose levels in patients with difficult-to-control (DTC) or resistant hypertension (RH).

### Methodology

Five cohorts of eight hypertensive subjects on stable doses of at least three hypertensive drugs (including a diuretic) for at least 6 weeks prior to screening are selected. Subjects are evaluated for participation in the study during a 3-week Screening Period including a blood pressure (BP) Qualifying Visit, 2 to 5 days prior to randomization. Eligible subjects are admitted to the Pharmacokinetic (PK) Unit for 7 days with 5 days of treatment administration followed by two safety follow-up visits on Days 12 and 21 (Tables 1-3). The total length of participation is approximately 42 days.

Approximately 45 adult male and female subjects between the ages of 18 and 80 years with DTC/RH are planned to be analyzed, with 37 patients randomized (28 to five successive cohorts and 9 to placebo). The study aims to enroll 50% African Americans (AA) and 50% Non-African Americans (NAA). Subjects are randomized in a 6:2 ratio to one of the 5 cohorts.

MANP disclosed herein (SEQ ID NO: 1) is tested in 5 cohorts once daily for 5 days. Cohort 1 receives doses of 3 μg/kg MANP. Cohort 2 receives 4 μg/kg MANP. Cohort 3 receives 4.5 μg/kg MANP. Cohort 4 receives 7 μg/kg MANP. Cohort 5 receives 6 μg/kg MANP. Each dose of the study treatment (MANP or placebo) is administered as a single subcutaneous bolus injection in the morning at approximately 8 am (±1 hour) for 5 consecutive days. Placebo is 0.9% saline solution administered as a single subcutaneous bolus injection in the morning at approximately 8 am (±1 hour) for 5 consecutive days. The treatment assignments were double-blind for the duration of the study.

### Inclusion Criteria:

Patients meet all of the following inclusion criteria:
1. Patient has DTC/RH, defined as hypertensive subjects with clinical systolic blood pressure systolic blood pressure (SBP)≥140 mmHg or diastolic blood pressure diastolic blood pressure (DBP)≥90 mmHg (or SBP≥130 mmHg or DBP≥80 mmHg for diabetics) derived from the average BPs from the Screening Visit and Visit 1, while on at least three standard-of-care antihypertensive medications (which must include a diuretic) for at least 6 weeks prior to Screening. Mean BPs at each visit are derived from the average of two seated BPs measured with an Indie Health Blood Pressure (BP) Monitor after the subject had been at rest in the seated position for at least three minutes and with the cuff remained in position but completely deflated for a full minute between measurements.
2. Patient has an estimated glomerular filtration rate (eGFR) calculated based on the equation from the Modification of Diet in Renal Disease Study (MDRD) MDRD eGFR≥30 mL/min/1.73 m².
3. Patient is between the ages of 18 and 80 years.
4. Patient has a body mass index (BMI) of 18-40 kg/m².
5. Female patient of childbearing potential has had a negative serum pregnancy test result at Screening and a negative urine pregnancy test on admission to the PK Unit at Visit 2, Day −1.
(i) Female patient who is not of childbearing potential is defined as:
   a) Postmenopausal (defined as at least 12 months with no menses in women≥45 years of age) with documented blood follicle stimulating hormone (FSH)>40 mIU/mL; OR
   b) Has had a hysterectomy and/or bilateral oophorectomy, bilateral salpingectomy, or bilateral tubal ligation/occlusion at least 6 weeks prior to Screening; OR
   c) Has a congenital or acquired condition that prevented childbearing.
(ii) Female patient of childbearing potential agrees to avoid becoming pregnant while receiving study treatment and for 14 days after the last study visit by complying with one of the following:
   a) practiced abstinence from heterosexual activity, OR
   b) uses (or had her partner use) acceptable contraception during heterosexual activity. Acceptable methods of contraception are:
      A. hormonal contraception (contraceptive pill or injection); and an additional barrier method of contraception such as a diaphragm, condom, sponge, or spermicide.
      B. vasectomy of a female subject's male partner.
      C. in the absence of hormonal contraception or vasectomy of a female subject's male partner, combination contraceptives methods (requiring use of two of the following) are used:
         a. non-hormonal intrauterine device (IUD);
         b. diaphragm with spermicide (could not be used in conjunction with cervical cap/spermicide);
         c. cervical cap with spermicide (nulliparous women only);
         d. contraceptive sponge (nulliparous women only);
         e. male condom or female condom (could not be used together).
6. Males willing to use a barrier birth control method (condom) for ANY sexual activity throughout the study and for an additional 14 days after the last study visit (Day 21).
7. Patient is able to communicate effectively with the study personnel.
8. Patient is willing and able to comply with the study procedures and visit schedule including follow-up visits.
9. Patient is adequately informed of the nature and risks of the study and gives written informed consent prior to receiving study medication in languages relevant to the study population.

### Exclusion Criteria:

Subjects who meet any of the following criteria are excluded from the study:
1. Has known hypersensitivity or allergy to MANP or its components, carperitide, other natriuretic peptides, or related compounds.
2. Has mean orthostatic BP changes derived from the average orthostatic changes from the Screening Visit and Visit 1 defined as a mean decrease in SBP of >20 mmHg or a mean decrease in DBP of >10 mmHg.
3. Has orthostatic symptoms (e.g., dizziness, light headedness, and/or palpitations) within two minutes of standing from the sitting position at either the Screening Visit or Visit 1.
4. Has HbA1c≥8% at Screening.
5. Subject is pregnant or breastfeeding.
6. Has any disease or condition (medical or surgical) which, in the opinion of the Investigator, might compromise the hematologic, cardiovascular, pulmonary, renal, gastrointestinal, hepatic, or central nervous system; or other conditions that may interfere with the absorption, distribution, metabolism or excretion of study drug, or would place the subject at increased risk.
7. Has completed of a COVID-19 vaccination regimen within 30 days of screening. Subjects who complete a vaccination regimen more than 30 days before screening are eligible for participation. Completion of a vaccination regimen refers to the last injection of the recommended vaccination regimen.
8. Non COVID-19 vaccinated subjects who do not agree to refrain from receiving vaccination during participation in this study.
9. Has a history of unstable angina or myocardial infarction within 6 months prior to Screening.
10. Has New York Heart Association (NYHA) Grade 3 or 4 congestive heart failure (CHF), valvular heart disease, ventricular cardiac arrhythmia requiring treatment or pulmonary hypertension.
11. Has cardiac surgery, coronary angioplasty, stroke, or transient ischemic attack (TIA) within 6 months prior to Screening.
12. Has abnormal laboratory values at the Screening Visit that is considered clinically significant by the Investigator. However, subjects are excluded if a) Serum sodium of <135 mEq/L or >145 mEq/L; b) Serum potassium of <3.5 mEq/L or >5.5 mEq/L.
13. Has a positive screen for Hepatitis B (HbsAg, Hepatitis B Surface Antigen), Hepatitis C (anti HCV, Hepatitis C Antibody), or HIV (anti-HIV 1/2).
14. Receives an Investigational drug within a 30-day period of the Screening Visit including the COVID-19 vaccine.
15. Subject has a recent history (within 1 year of Screening) of alcohol abuse or dependence. Alcohol abuse includes heavy alcohol intake as defined by >3 drinks per day or >14 drinks per week or binge drinking.
16. Has consumed alcohol within 72 hours prior to dose administration or during any in-patient period.
17. Has a positive urine drug screen including ethanol, cocaine, THC, barbiturates, amphetamines, benzodiazepines, and opiates unless, in the opinion of the Investigator, a positive finding as a result of a legitimate medical prescription for a valid medical condition. Occasional intermittent use of cannabinoid products is allowed provided that no cannabinoid products are used during the 1 week prior to each visit.
18. Has a history (within the last 2 years) of illicit drug use, significant mental illness, physical dependence to any opioid, or any history of drug abuse or addiction.
19. Has a history of difficulty with donating blood or donated blood or blood products within 45 days prior to enrollment.
20. Has a clinically significant new illness in the 1 month before Screening in the opinion of the Investigator.
21. Has a history of severe allergies (e.g., anyone with a known history of anaphylaxis to medication[s] or allergens and/or asthma requiring hospitalization).
22. Has malignancy within 5 years of the Screening visit (with the exception of basal and squamous cell skin carcinoma and treated carcinoma-in-situ (CIS) of the cervix).
23. Has known prior exposure to any ANP-related peptide (MANP, nesiritide, carperitide, or other ANP-related peptides in development), whether previously FDA-approved or not.
   Removal of Subjects from Dosing or Assessment:

Subjects who are discontinued from dosing or from the study for any of the following reasons:
1. During the Ascending Dose Phase of the trial, dose escalation is stopped if 2 subjects in the cohort develops clinically significant hypotension, defined as a decrease in sitting SBP to <90 mmHg confirmed by repeat measurement, or accompanying lightheadedness or dizziness or visual symptoms. Once the stopping criteria are met, a decision is made by the Investigator and sponsor to enter one or more additional cohorts at the same dosage level (in the absence of serious toxicity) or at a lower dosage level, but higher doses were not to be administered.
2. The subject experiences an adverse event (AE) that in the judgement of the Investigator is related to the Investigational drug and poses a significant risk to the subject for continued participation in the study.
3. The subject becomes pregnant.
4. There is significant protocol violation or noncompliance on the part of the subject or the Investigator.
5. There is intercurrent illness that requires treatment not consistent with the protocol requirements, or intercurrent illness and the associated treatment poses a significant risk to the subject for continued participation in the study in the judgment of the Investigator.
6. The subject meets one of the exclusion criteria during the study.
7. The subject wishes to withdraw for any reason.
8. The sponsor elects to end the study, or the Investigational Site elects to end the study at their site.
9. There is any other reason that in the judgment of the Investigator poses an unacceptable risk to the subject.

### Concomitant Therapy:

All medications and supplements (other than IP) taken by the subject from Visit 2, Day 1 through Visit 4 are considered “concomitant” medications and supplements. Medications and supplements taken prior to Visit 2, Day 1 that are no longer being taken at the time of Visit 2, Day 1 are considered “prior” medications and supplements.

Clinical BPs and Orthostatic BPs:

Clinical BPs are measured with an Indie Health BP Monitor at Screening, Visit 1, Day-1, pre-dose (−20 min) on Day 1 (±15 min), and at 0.5, 1, 2, 4, 6, 12 hours (±5 min), and 24 hours (±15 min) post-dose on Days 1-5, Visits 3 and 4, and ET Visit.

Clinical BPs are measured after the subject had been resting for at least 3 minutes and are measured prior to any blood draw that occurred at the same time point. Clinical BPs are derived from the average of two BPs measured with an Indie Health BP Monitor after the subject had been at rest in the seated position for at least three minutes and with the cuff remaining in position but completely deflated for a full minute between measurements.

Orthostatic BPs are measured with an Indie Health BP Monitor. Subjects are seated for 3 min, and BP is measured. The subjects are made to stand, and a single BP measurement is taken 2 min after standing. Orthostatic BPs are measured at Screening, Visit 1, pre-dose (−20 min) on Day 1 (±15 min), and at 0.5, 1, and 6 hours (±5 min) post-dose on Days 1-5, Visits 3 and 4, and ET Visit.

TABLE 1
Daily schedule of events (Screening through Safety Follow-up Visit 4)

| | Screening Period | | PK Unit Assessments | | | |
|---|---|---|---|---|---|---|
| | | BP Qualifying | Admission | Treatment | Safety | Safety |
| | | Visit | to PK Unit | Administration | Follow-up | Follow-up |
| | Visit | | | | | |
| | Screening | | | | | |
| | Visit | Visit 1 | Visit 2 | | Visit 3 | Visit 4 |
| | Study Day | | | | | |
| | Days −21 | | | | Day 12 ± | Day 21 ± |
| | to −7 | Days −5 to −2 | Day −1 | Days 1-6 | 3 | 3 |
| PK Unit | | | X | X | | |
| Admit/Discharge | | | | | | |
| Clinical BPs | X | X | X | X | X | X |
| Orthostatic BPs | X | X | | X | X | X |
| Plasma cGMP | | | | X | | |
| Plasma | | | | X | X | X |
| aldosterone | | | | | | |
| Creatinine for | X | | | | | X |
| eGFR | | | | | | |
| Study Drug | | | | X | | |
| Administration | | | | | | |

TABLE 2
PK Unit Schedule of Events (Admission
to PK Unit, Visit 2, Day −1)

| Day | Time | Clinical BP |
|---|---|---|
| −1 | 18:00-20:00 | X |

TABLE 3
PK Unit Schedule of Evens (Visit 2, Days 1-6)

| | | | Administer | | | Plasma | Plasma | |
|---|---|---|---|---|---|---|---|---|
| | | | Investigational | | | cGMP | aldosterone | |
| | | Elapsed | Product | Clinical | Orthostatic | blood | blood | Additional |
| Day | Time | Hour | Subcutaneous | BP | BP | draw | draw | Comments |
| 1 | 07:40 | | | X | X | X | X^{buffy} | Baseline pre- |
| | | | | | | | | dose |
| | | | | | | | | measurement ~20 |
| | | | | | | | | minutes |
| | | | | | | | | prior to |
| | | | | | | | | dosing. |
| | 08:00 | 0 | X | | | | | |
| | 08:15 | 0.25 | | | | X | | |
| | 08:30 | 0.5 | | X | X | X | | |
| | 09:00 | 1 | | X | X | X | X | |
| | 09:30 | 1.5 | | | | X | | |
| | 10:00 | 2 | | X | | | | |
| | 11:00 | 3 | | | | X | X | |
| | 12:00 | 4 | | X | | | | |
| | 14:00 | 6 | | X | X | X | X | |
| | 20:00 | 12 | | X | X | | X | |
| 2 | 7:40 | | | X | | | | |
| | 08:00 | 24 | X | | | | | |
| | 08:30 | 24.5 | | X | X | | | |
| | 09:00 | 25 | | X | X | | | |
| | 10:00 | 26 | | X | | | | |
| | 12:00 | 28 | | X | | | | |
| | 14:00 | 30 | | X | X | | | |
| | 20:00 | 36 | | X | | | | |
| 3 | 7:40 | | | X | | | | |
| | 08:00 | 72 | X | | | | | |
| | 08:30 | 72.5 | | X | X | | | |
| | 09:00 | 73 | | X | X | | | |
| | 10:00 | 74 | | X | | | | |
| | 12:00 | 76 | | X | | | | |
| | 14:00 | 78 | | X | X | | | |
| | 20:00 | 84 | | X | | | | |
| 4 | 7:40 | | | X | | | | |
| | 08:00 | 96 | X | | | | | |
| | 08:30 | 96.5 | | X | X | | | |
| | 09:00 | 97 | | X | X | | | |
| | 10:00 | 98 | | X | | | | |
| | 12:00 | 100 | | X | | | | |
| | 14:00 | 102 | | X | X | | | |
| | 20:00 | 108 | | X | | | | |
| 5 | 7:40 | | | X | | X | X | |
| | 08:00 | 96 | X | | | | | |
| | 08:15 | 96.25 | | | | X | | |
| | 08:30 | 96.5 | | X | X | X | | |
| | 09:00 | 97 | | X | X | X | X | |
| | 09:30 | 97.5 | | | | X | | |
| | 10:00 | 98 | | X | | | | |
| | 11:00 | 99 | | | | X | X | |
| | 12:00 | 100 | | X | | | | |
| | 14:00 | 102 | | X | X | X | X | |
| | 20:00 | 108 | | X | | X | X | |
| 6 | 7:40 | | | X | | X | X^{buffy} | |

eGFR:

Serum chemistry is measured and the amount of serum creatine is measured at Screening (baseline) and at Visit 4 on Day 21. The estimated Glomerular Filtration Rate (eGFR) and change from baseline are calculated and summarized.

Cyclic Guanosine Monophosphate (cGMP):

Levels of plasma cyclic guanosine monophosphate (cGMP) are determined. Samples are collected pre-dose (−20 min) (±15 min), and at 0.25, 0.5, 1, 1.5, 3, 6, 12 hours (±5 min), and 24 hours (±15 min) post-dose on Day 1 and Day 5 (see Table 3). Approximately 108 mL of blood is collected for pharmacokinetic samples. The plasma samples are shipped on dry ice via overnight delivery to the analytical lab for analysis.

### Aldosterone:

Levels of plasma aldosterone are determined. Samples are collected pre-dose (−20 min) (#15 min), and at 1, 3, 6, 12 hours (±5 min), and 24 hours (±15 min) post-dose on Day 1 and Day 5, Visits 3 and 4 (see Table 3). The Buffy coat from the Day 1 pre-dose sample and Day 6 sample is retrieved and stored. The plasma samples are shipped on dry ice via overnight delivery to the analytical lab for analysis.

### Statistical and Analytical Plan:

All measured variables and derived parameters are listed individually and tabulated by descriptive statistics. For descriptive statistics, summary tables are provided giving sample size, frequency (counts and percentages) for proportions of categorical variables by study group and sample size, arithmetic mean, standard deviation, coefficient of variation (if appropriate), median, minimum, and maximum, by study group for continuous variables.

Prior and concomitant medications will be coded using the World Health Organization (WHO) Drug dictionary September 2021 version.

Clinical BPs are measured at Screening, Visit 1, Visit 2 Day-1, Visit 2 Day 1 pre-dose (−20 min) on Day 1 (±15 min), and at 0.5, 1, 2, 4, 6, 12 hours (±5 min), and 24 hours (±15 min) post-dose on Days 1-5, Visit 3, and Visit 4. Individual values are listed and summarized using descriptive statistics as appropriate (i.e., mean, median, range, and standard deviation). Individual change from baseline (Pre-dose Visit 2, Day 1) clinical BP values are calculated and summarized descriptively.

Percentage change toward ideal values for SBP, DBP, and Pulse Rate are listed and summarized descriptively by visit and timepoint and a responder rate of subjects achieving the ideal value is also be listed and summarized by visit and timepoint using frequencies and percentages. Percentage change toward ideal value is defined as the following:

$$1 - ( \frac{Post - dose value - Ideal value}{Pre - dose value - Ideal value} )$$

This statistic is a measure of how close subjects' post-dose value is to the ideal value, relative to how much of a drop was needed from their Pre-dose value to achieve the ideal value.

If a Post-dose value is less than or equal to the Pre-dose value, then the result is:
Between 0% and 100% when the Post-dose SBP value is more than the ideal value,
Exactly 100% when the post-dose value equals the ideal value, and
More than 100% when the post-dose value is less than the ideal value.

If the Post-dose value is greater than the Pre-dose value, then the result is negative. Ideal values of SBP and DBP of 110 mmHg and 70 mmHg respectively, are used.

For Orthostatic BP, subjects are seated for 3 min, and BP is measured. The subjects are made to stand, and a single BP measurement is taken 2 min after standing. Orthostatic BPs are measured pre-dose (−20 min) on Day 1 (±15 min), and at 0.5, 1, and 6 hours (±5 min) post-dose on Days 1-5.

Individual values are listed and summarized using descriptive statistics as appropriate (i.e., mean, median, range, and standard deviation). Individual change from baseline (Predose Visit 2, Day 1) values are calculated and summarized descriptively.

The eGFR is calculated and summarized using descriptive statistics as appropriate (i.e., mean, median, range, and standard deviation). The eGFR change from baseline is calculated and summarized descriptively.

Pharmacokinetic and pharmacodynamic analysis is conducted using model-independent methods as implemented in Phoenix WinNonlin® (version 8.0 or later) and are based on plasma concentrations of cGMP. Individual plasma concentrations of cGMP are listed for each subject and sampling time and summarized descriptively using the arithmetic mean, SD, CV (%), median, minimum, and maximum. Individual plasma concentration-time profiles of cGMP are plotted on both a linear and a semi-logarithmic scale. Mean values are also presented graphically. Pharmacokinetic and pharmacodynamic parameters of cGMP are listed for each subject. cGMP is present in the subjects regardless of treatment. To correct for this, both the observed and change from pre-dose values are analyzed.

### Results—Patient Demographics

A summary of subject disposition comparing MANP and placebo for all subjects is presented in Table 4.

TABLE 4
Summary of Subject Disposition - All Subjects

| | Placebo | 3 μg/kg | 4 μg/kg | 4.5 μg/kg | 7 μg/kg | 6 μg/kg |
|---|---|---|---|---|---|---|
| | (N = 9) | (N = 6) | (N = 6) | (N = 6) | (N = 4) | (N = 6) |
| Subjects | 9 | 6 | 6 | 6 | 4 | 6 |
| Randomized | | | | | | |
| Completed | 9 (100) | 6 (100) | 6 (100) | 6 (100) | 3 (75.0) | 6 (100) |
| the Study - | | | | | | |
| n (%) | | | | | | |
| Discontinued | 0 (0.0) | 0 (0.0) | 0 (0.0) | 0 (0.0) | 1 (25.0) | 0 (0.0) |
| the Study - | | | | | | |
| n (%) | | | | | | |

Reason for Discontinuation from Study - n (%)

| | | | | | | |
|---|---|---|---|---|---|---|
| Adverse | 0 (0.0) | 0 (0.0) | 0 (0.0) | 0 (0.0) | 1 (25.0) | 0 (0.0) |
| event | | | | | | |

A summary of subject demographics and baseline characteristics for all subjects is presented in Table 5.

TABLE 5
Summary of Subject Demographics and Baseline Characteristics - All Subjects

| | | | | | |
|---|---|---|---|---|---|
| Placebo | 3 μg/kg | 4 μg/kg | 4.5 μg/kg | 7 μg/kg | 6 μg/kg |
| (N = 9) | (N = 6) | (N = 6) | (N = 6) | (N = 4) | (N = 6) |

Sex - n (%)

| | | | | | | |
|---|---|---|---|---|---|---|
| Male | 5 (55.6) | 4 (66.7) | 3 (50.0) | 2 (33.3) | 2 (50.0) | 0 |
| Female | 4 (44.4) | 2 (33.3) | 3 (50.0) | 4 (66.7) | 2 (50.0) | 6 (100) |

Race - n (%)

| | | | | | | |
|---|---|---|---|---|---|---|
| White | 4 (44.4) | 2 (33.3) | 3 (50.0) | 2 (33.3) | 3 (75.0) | 3 (50.0) |
| Black or | 5 (55.6) | 3 (50.0) | 3 (50.0) | 3 (50.0) | 1 (25.0) | 3 (50.0) |
| African | | | | | | |
| American | | | | | | |
| Asian | 0 | 1 (16.7) | 0 | 0 | 0 | 0 |
| American | 0 | 0 | 0 | 0 | 0 | 0 |
| Indian or | | | | | | |
| Alaskan | | | | | | |
| Native | | | | | | |
| Native | 0 | 0 | 0 | 1 (16.7) | 0 | 0 |
| Hawaiian or | | | | | | |
| Other Pacific | | | | | | |
| Islander | | | | | | |
| Other | 0 | 0 | 0 | 0 | 0 | 0 |
| Not Reported | 0 | 0 | 0 | 0 | 0 | 0 |

Ethnicity - n (%)

| | | | | | | |
|---|---|---|---|---|---|---|
| Hispanic or | 1 (11.1) | 0 | 0 | 0 | 0 | 0 |
| Latino | | | | | | |
| Not Hispanic | 8 (88.9) | 6 (100) | 6 (100) | 6 (100) | 4 (100) | 6 (100) |
| or Latino | | | | | | |
| Not Reported | 0 | 0 | 0 | 0 | 0 | 0 |
| Not Required | 0 | 0 | 0 | 0 | 0 | 0 |

Age (years)

| | | | | | | |
|---|---|---|---|---|---|---|
| Sample Size | 9 | 6 | 6 | 6 | 4 | 6 |
| Mean | 59.03 | 61.60 | 64.25 | 63.65 | 59.13 | 60.28 |
| Standard | 7.723 | 13.882 | 6.229 | 4.780 | 9.305 | 7.812 |
| Deviation | | | | | | |
| Median | 60.10 | 64.40 | 63.05 | 64.75 | 61.80 | 58.90 |
| Min, Max | 47.4, 70.6 | 35.3, 76.9 | 56.5, 73.9 | 55.2, 68.7 | 45.7, 67.2 | 50.7, 73.3 |

BMI (kg/m²) at Screening

| | | | | | | |
|---|---|---|---|---|---|---|
| Sample Size | 9 | 6 | 6 | 6 | 4 | 6 |
| Mean | 32.000 | 36.017 | 34.500 | 30.483 | 31.125 | 36.433 |
| Standard | 3.4322 | 2.5031 | 5.0323 | 4.0177 | 5.3056 | 10.1774 |
| Deviation | | | | | | |
| Median | 30.700 | 35.550 | 35.650 | 29.100 | 31.550 | 33.050 |
| Min, Max | 27.50, 37.40 | 32.70, 39.90 | 28.30, 39.40 | 26.30, 36.20 | 24.30, 37.10 | 26.40, 55.20 |

Results—Change in Sitting Systolic BP from Baseline

For study qualification, DTC/RH subjects have clinic SBP≥140 mmHg or DBP≥90 mmHg (or SBP≥130 mmHg or DBP≥80 mmHg for diabetics) derived from the average BPs from Screening and Visit 1, while on at least three standard-of-care antihypertensive medications which must include a diuretic.

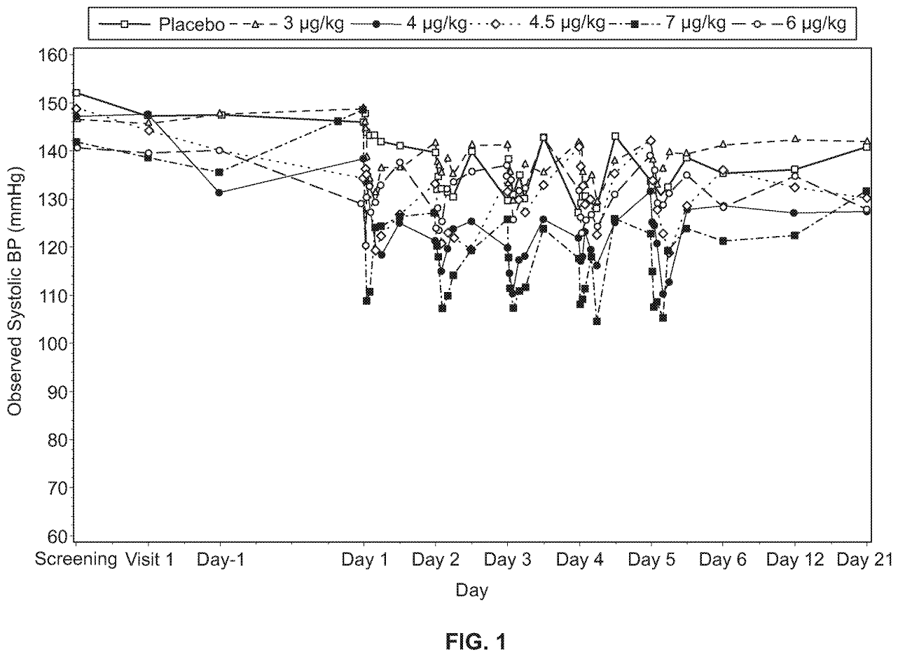

The average qualifying Screening and Visit 1 SBPs per treatment group are depicted in the first two data points (from the left) in FIG. 1. The placebo group has the highest screening SBPs (152.1 mmHg) that were 4.4 mmHg higher than 3 μg/kg group (146.7 mmHg), 5.1 mmHg higher than 4 μg/kg group (147.0 mmHg), 3.3 mmHg higher than 4.5 μg/kg group (148.8 mmHg), 10.3 mmHg higher than 7 μg/kg group (146.7 mmHg), and 11.4 mmHg higher than 6 μg/kg group (140.7 mmHg). These differences generally persist through Visit 1 and Day 1 when study treatment dosing commenced. While the Screening and Visit 1 SBPs are typical daytime measures, the Day-1 SBPs are collected in the evening on the admission day approximately 12 hours before the administration of the first dose on Day 1 and cannot be reliably compared to the Screening, Visit 1 and Day 1 baseline SBPs.

MANP dosing is administered in the morning (8 am±1-hour) on Days 1 through 5 and ends on the morning of Day 6. On each dosing day, BPs and pulse rate are collected at 0.5, 1, 2, 4, 6, 12 and 24 hours after dosing and are represented by corresponding data point on Days 1 through 5 in FIG. 1. Thus, Days 1 through 5 each have 7 data points.

While each dose group is small (N=6 for all, except N=4 for the 7 μg/kg group) and had variable BPs with considerable overlap, the SBPs of the 7, 4, and 4.5 μg/kg groups generally clusters in the lower ranges of SBPs while the placebo, 3, and 6 μg/kg groups generally has the higher SBPs. These changes are discussed in more detail below.

The SBPs on Days 12 and 21 (on the right side of FIG. 1) are collected at safety follow-up visits approximately 7 and 16 days after the last dose on MANP on the morning of Day 5.

Interestingly, despite the absence of MANP over this period, these SBPs are lower than the Screening SBPs by 11.2 mmHg in the placebo group and by 4.9, 19.5, 18.5, 10, and 12.7 mmHg in the 3, 4, 4.5, 7, and 6 μg/kg groups respectively which may reflect the lingering antihypertensive effects of MANP.

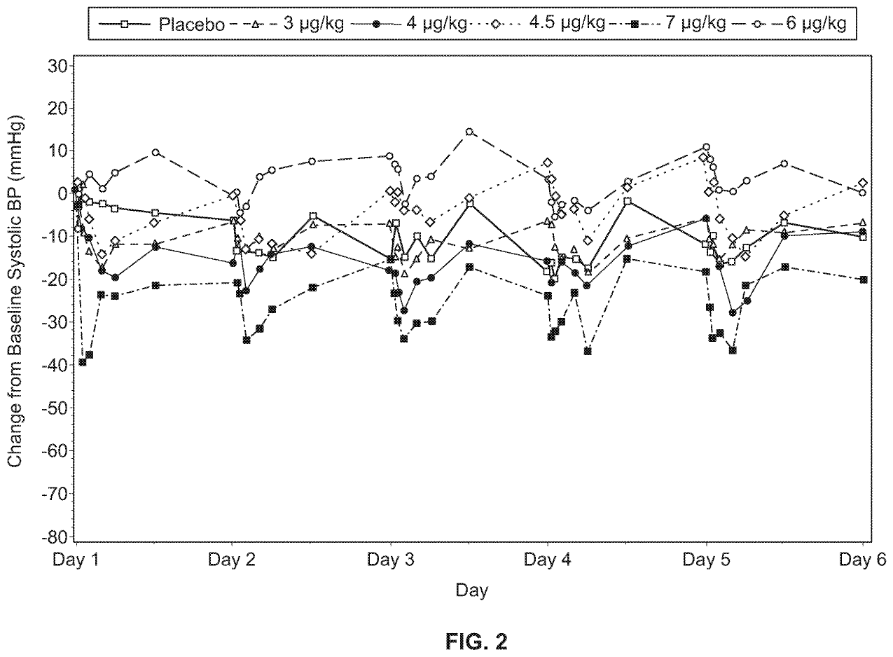

FIG. 2 represents the average changes in sitting SBP for all treatment groups from baseline over the 5 days of therapy, commencing on the morning of Day 1 and concluding on the morning of Day 6, twenty-four hours after the last MANP dose on the morning of Day 5.

For the purposes of assessing SBP effects, baseline SBP is determined as the Visit 2 Day 1 Pre-dose value, typically assessed at 8 am±1 hour just prior to the first dose of subcutaneous MANP or Placebo on Visit 2 Day 1. Thereafter, subsequent doses of MANP or placebo are administered at approximate 24-hour intervals on Visit 2 Day 2, Visit 2 Day 3, Visit 2 Day 4, and Visit 2 Day 5.

As is the case for Screening and Visit 1 SBPs, baseline SBPs (Table 6) differs considerably between the treatment groups with the placebo (145.9 mmHg), 3 μg/kg (148.8 mmHg), and the 7 μg/kg (148.5 mmHg) having the highest baseline SBPs, while the 4 μg/kg (138.3 mmHg), 4.5 μg/kg (134.3 mmHg), and the 6 μg/kg (129.0 mmHg) groups have lower baseline SBPs that are respectively 7.6 mmHg, 11.6 mmHg, and 16.9 mmHg less than the placebo baseline SBP.

In considering the changes in SBP from baseline depicted in FIG. 2, the 7 μg/kg group clearly has the greatest SBP reductions, and while there is considerable overlap among the remaining groups, the 4 μg/kg group generally generates the next largest SBP reductions. Exhibited strikingly in the 7 μg/kg group, but generally present for all groups, SBP falls after each morning dose attaining a peak reduction around 6 hours after dosing and then gradually diminishes thereafter.

Table 6 tabulates the baseline SBP along with the maximum and minimum SBP changes and their associated time points for each treatment group for each of the 5 days of therapy. Aside from the placebo group which is a compilation of the placebo treated subjects from each treatment group, the order of the treatment group columns from left to right represents the sequence of ascending doses from 3 μg/kg to 7 μg/kg, and then finally, the reduction to 6 μg/kg.

The placebo treatment group has large reductions in SBP which is probably a function of higher baseline BPs and the general sedentary nature associated with in-house confinement. Peak placebo SBP reductions mostly occurs 24 hours after dosing after the greatest number of sedentary hours associated with the prior night's sleep and rest.

Considering the maximum SBP reductions for the remaining treatment groups, especially on Day 1, there appears to be a dose-response relationship with increasing SBP reductions as one progresses from 3 μg/kg to 4 μg/kg, and then to 7 μg/kg. While the 4.5 μg/kg generally has lower reductions than the 4 μg/kg group, probably related to its lower baseline starting point, considering the 4 and 4.5 μg/kg groups as a single entity does not detract from the general sense of a dose-response relationship as one progresses from 3 μg/kg, through the combined 4 and 4.5 μg/kg groups to the 7 μg/kg group.

The SBP reductions for the 6 μg/kg group are oddly the lowest of the treatment groups, but it must be appreciated that this group's starting baseline is 11.4 mmHg less than the placebo baseline and that if this difference is hypothetically added to the results of the 6 μg/kg group the general sense of a dose-response relationship is maintained-especially on Day 1.

For each treatment group, peak SBP reductions (Max 4) occur within the first 6 hours of dosing, while minimum SBP reductions (Min A) generally occur beyond the 12-hour timepoint or very early in the next dosing cycle before the onset of activity of the next dose. Thus, peak SBP reductions clusters around the 4-to-6-hour timepoints after dosing while minimum reductions clustered around the latter part of the 24-hour dosing cycle.

TABLE 6
Baseline SBP along with the maximum and minimum
SBP changes (Δ) and their associated time points
(hours after dosing) for each of the 5 days of
therapy - All Subjects (Safety Population)

| | | Pla- | 3 | | | | |
|---|---|---|---|---|---|---|---|
| | | cebo | μg/kg | 4 | 4.5 | 7 | 6 |
| | | (N = | (N = | μg/kg | μg/kg | μg/kg | μg/kg |
| | | 9) | 6) | (N = 6) | (N = 6) | (N = 4) | (N = 6) |
| Dose | Baseline | 145.9 | 148.8 | 138.3 | 134.3 | 148.5 | 129.0 |
| Day | (mmHg) | | | | | | |
| Day | Max Δ | −6.3 | −17.5 | −20.0 | −15.0 | −39.5 | −8.7 |
| 1 | Timepoint | 24 h | 4 h | 6 h | 4 h | 1 h | 0.5 h |
| | Min Δ | 2.1 | −3.8 | −3.2 | 1.8 | −14.0 | 8.7 |
| | Timepoint | 0.5 h | 0.5 h | 0.5 h | 0.5 h | 0.5 h | 12 h |
| Day | Max Δ | −16.1 | −13.7 | −23.2 | −14.5 | −34.3 | −5.0 |
| 2 | Timepoint | 24 h | 6 h | 2 h | 12 h | 2 h | 1 h |
| | Min Δ | −6.0 | −7.7 | −12.2 | 0.0 | −15.7 | 8.0 |
| | Timepoint | 12 h | 24 h | 1 h | 24 h | 24 h | 24 h |
| Day | Max Δ | −18.6 | −19.3 | −27.8 | −7.2 | −34.3 | −3.3 |
| 3 | Timepoint | 24 h | 2 h | 2 h | 6 h | 2 h | 2 h |
| | Min Δ | −3.0 | −7.2 | −12.5 | 6.5 | −17.7 | 13.7 |
| | Timepoint | 12 h | 24 h | 12 h | 24 h | 12 h | 12 h |
| Day | Max Δ | −20.0 | −19.2 | −21.8 | −11.8 | −37.0 | −6.0 |
| 4 | Timepoint | 1 h | 6 h | 6 h | 6 h | 6 h | 1 h |
| | Min Δ | −2.6 | −6.5 | −6.7 | 7.8 | −15.7 | 10.0 |
| | Timepoint | 12 h | 24 h | 24 h | 24 h | 12 h | 24 h |
| Day | Max Δ | −16.7 | −16.0 | −28.0 | −15.5 | −36.3 | −0.5 |
| 5 | Timepoint | 2 h | 2 h | 4 h | 6 h | 4 h | 24 h |
| | Min Δ | −7.3 | −7.5 | −9.7 | 2.0 | −17.7 | 7.3 |
| | Timepoint | 12 h | 24 h | 24 h | 1 h | 12 h | 0.5 h |

Baseline is defined as Visit 2 Day 1 Pre-Dose. SBP changes (A)=Post-Dose SBP value-Visit 2 Day 1 Pre-Dose Baseline. Negative values indicate an SBP reduction; positive values an SBP increase. Thus, maximum changes (Max A) represent the largest SBP reductions (i.e., largest negative values) or the smallest positive value if all changes were positive (i.e., SBP increased). Minimum changes (Min A) represent the smallest SBP reductions (i.e., smallest negative values) or the largest positive value if all changes were positive (i.e., SBP increased).

This study is designed to evaluate the percentage change toward ideal BP with the ideal values set at 110 mmHg, 70 mmHg for SBP and DBP respectively.

If a post-dose value is less than or equal to the pre-dose value, then the percentage toward the ideal value would be between 0% and 100% depending on the degree to which the reduction from the baseline value approaches the ideal value. If the reduction reaches or exceeds the ideal value, the associated percentage change would be 100% or >100% respectively. In instances where post-dose values exceed pre-dose values the percentage toward ideal generates a negative value.

Along with SBP reductions, these percentage toward ideal values are tabulated in Table 6 for each timepoint of the 5 days of dosing. As a result of the small groups and the fluctuation of SBP this metric generates a wide array of values. However, generally speaking, the maximum SBP reductions attain a percentage toward ideal of between 20% to 100% for the 3, 4, 4.5, 7, and 6 μg/kg groups.

Finally, it is noted that these SBP reductions and the percentage toward ideal SBPs are attained in DTC/RH subjects requiring at least three standard-of-care antihypertensive medications that includes a diuretic.

Results—Changes in cGMP Levels from Baseline

The cGMP levels by timepoint for all subjects is shown in Table 7. The summary of cGMP pharmacokinetic parameters for all subjects is shown in Table 8.

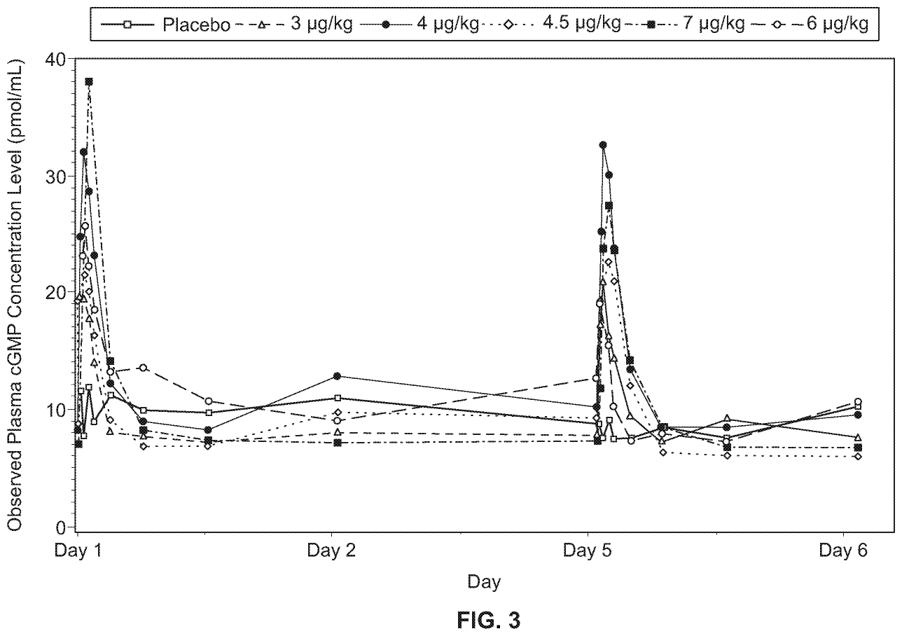

While cGMP values are comparable at baseline on both Days 1 and 5, the different doses of MANP yields clear peak levels early in the dosing cycle, while placebo does not produce a clearly discernable peak level. The 3 ug/kg dose leads to peak levels of 19.70 and 20.75 μmol/mL at hours 0.25 and 0.5 on days 1 and 5 respectively; the 4 μg/kg dose to peak levels of 31.97 and 32.60 μmol/mL at hours 0.5 and 0.5 on Days 1 and 5 respectively; the 4.5 μg/kg dose to peak levels of 21.85 and 22.66 μmol/mL at hours 0.5 and 1 on Days 1 and 5 respectively; the 7 μg/kg dose to peak levels of 37.97 and 27.50 μmol/mL at hours 1 and 1 on Days 1 and 5 respectively; and the 6 μg/kg dose to peak levels of 25.68 and 18.98 μmol/mL at hours 0.5 and 0.25 on Days 1 and 5 respectively. Placebo produces the highest levels of 12.02 and 9.03 μmol/mL at hours 1 and 1 on Days 1 and 5 respectively. Thus, the 7 μg/kg dose group produces the highest peak levels while the other doses ranges between that of placebo and 7 μg/kg (Table 7 and FIG. 3).

TABLE 7
cGMP Levels by Timepoint - All Subjects (PK Population)

| | Placebo | 3 μg/kg | 4 μg/kg | 4.5 μg/kg | 7 μg/kg | 6 μg/kg |
|---|---|---|---|---|---|---|
| | (N = 9) | (N = 6) | (N = 6) | (N = 6) | (N = 4) | (N = 6) |
| | cGMP Concentration (pmol/mL) | | | | | |
| Day 1: Pre-Dose | | | | | | |
| Sample Size | 9 | 6 | 6 | 6 | 4 | 6 |
| Mean | 9.79 | 8.33 | 10.83 | 8.92 | 7.23 | 10.00 |
| Standard Deviation | 4.384 | 3.217 | 5.074 | 5.265 | 1.466 | 6.460 |
| Median | 7.80 | 7.60 | 9.70 | 7.55 | 7.65 | 9.30 |
| Min, Max | 6.2, 19.9 | 5.6, 14.2 | 5.5, 19.3 | 4.7, 18.9 | 5.2, 8.4 | 3.6, 20.3 |
| Day 1: 0.25 | | | | | | |
| Hour Post-Dose | | | | | | |
| Sample Size | 9 | 6 | 6 | 6 | 3 | 6 |
| Mean | 11.57 | 19.70 | 24.87 | 19.30 | 18.80 | 23.22 |
| Standard Deviation | 7.292 | 11.249 | 9.735 | 11.063 | 13.098 | 8.667 |
| Median | 9.70 | 16.05 | 23.25 | 16.05 | 13.60 | 22.05 |
| Min, Max | 2.8, 26.7 | 9.2, 35.7 | 14.3, 41.4 | 11.3, 40.9 | 9.1, 33.7 | 13.4, 39.2 |
| Day 1: 0.5 | | | | | | |
| Hour Post-Dose | | | | | | |
| Sample Size | 9 | 6 | 6 | 6 | 3 | 6 |
| Mean | 7.93 | 19.35 | 31.97 | 21.85 | 27.77 | 25.68 |
| Standard Deviation | 4.403 | 8.975 | 12.775 | 15.807 | 12.671 | 11.697 |
| Median | 7.20 | 19.85 | 32.05 | 15.40 | 28.60 | 25.00 |
| Min, Max | 2.4, 18.1 | 7.6, 28.8 | 17.5, 49.4 | 10.5, 51.7 | 14.7, 40.0 | 13.1, 41.6 |
| Day 1: 1 Hour | | | | | | |
| Post-Dose | | | | | | |
| Sample Size | 9 | 6 | 6 | 6 | 3 | 6 |
| Mean | 12.02 | 17.72 | 28.53 | 20.13 | 37.97 | 22.20 |
| Standard Deviation | 6.313 | 10.600 | 13.619 | 19.849 | 16.523 | 14.626 |
| Median | 10.70 | 15.95 | 28.65 | 9.95 | 32.20 | 17.65 |
| Min, Max | 2.4, 20.6 | 7.5, 35.0 | 13.3, 42.9 | 7.0, 57.4 | 25.1, 56.6 | 8.9, 45.3 |
| Day 1: 1.5 | | | | | | |
| Hours Post-Dose | | | | | | |
| Sample Size | 9 | 6 | 6 | 6 | 3 | 6 |
| Mean | 9.04 | 13.95 | 23.15 | 16.38 | 28.23 | 18.50 |
| Standard Deviation | 4.780 | 8.517 | 9.149 | 14.956 | 14.479 | 10.709 |
| Median | 6.70 | 11.85 | 25.70 | 9.40 | 21.90 | 15.60 |
| Min, Max | 3.6, 17.3 | 4.1, 27.8 | 9.4, 32.3 | 5.1, 44.0 | 18.0, 44.8 | 8.5, 37.1 |
| Day 1: 3 Hours | | | | | | |
| Post-Dose | | | | | | |
| Sample Size | 9 | 6 | 6 | 6 | 3 | 5 |
| Mean | 11.26 | 8.18 | 12.28 | 9.18 | 14.20 | 13.22 |
| Standard Deviation | 5.176 | 3.820 | 5.449 | 5.132 | 1.609 | 6.934 |
| Median | 10.10 | 6.95 | 13.60 | 7.70 | 14.70 | 11.50 |
| Min, Max | 2.8, 18.8 | 4.7, 14.9 | 5.2, 17.9 | 3.9, 18.6 | 12.4, 15.5 | 5.8, 24.5 |
| Day 1: 6 Hours | | | | | | |
| Post-Dose | | | | | | |
| Sample Size | 9 | 6 | 6 | 6 | 4 | 5 |
| Mean | 9.98 | 7.77 | 9.02 | 6.93 | 8.28 | 13.68 |
| Standard Deviation | 7.890 | 2.435 | 6.574 | 2.361 | 0.981 | 14.007 |
| Median | 6.90 | 7.95 | 7.05 | 6.60 | 7.95 | 7.60 |
| Min, Max | 3.6, 28.8 | 5.0, 11.4 | 4.3, 22.2 | 3.9, 10.3 | 7.5, 9.7 | 3.2, 37.6 |
| Day 1: 12 Hours | | | | | | |
| Post-Dose | | | | | | |
| Sample Size | 9 | 6 | 5 | 6 | 3 | 6 |
| Mean | 9.78 | 7.23 | 8.36 | 6.98 | 7.43 | 10.80 |
| Standard Deviation | 5.286 | 2.547 | 3.299 | 4.688 | 0.231 | 5.285 |
| Median | 7.60 | 6.95 | 7.20 | 5.60 | 7.30 | 8.50 |
| Min, Max | 2.9, 20.1 | 4.0, 11.3 | 5.6, 14.0 | 3.7, 16.1 | 7.3, 7.7 | 7.7, 21.3 |
| Day 1: 24 Hours | | | | | | |
| Post-Dose | | | | | | |
| Sample Size | 9 | 6 | 5 | 6 | 4 | 6 |
| Mean | 11.08 | 8.12 | 12.94 | 9.83 | 7.25 | 9.08 |
| Standard Deviation | 6.358 | 2.461 | 5.415 | 7.256 | 2.779 | 4.166 |
| Median | 9.10 | 7.10 | 12.10 | 6.85 | 7.55 | 8.45 |
| Min, Max | 4.8, 25.7 | 5.8, 12.2 | 7.2, 21.6 | 4.7, 23.3 | 3.9, 10.0 | 4.2, 16.2 |
| Day 5: Pre-Dose | | | | | | |
| Sample Size | 9 | 6 | 6 | 6 | 3 | 6 |
| Mean | 8.76 | 7.90 | 10.38 | 9.38 | 7.37 | 12.78 |
| Standard Deviation | 4.781 | 4.912 | 6.098 | 7.207 | 1.665 | 10.528 |
| Median | 6.70 | 5.75 | 8.25 | 9.30 | 7.90 | 8.95 |
| Min, Max | 4.1, 19.6 | 4.7, 17.3 | 5.9, 22.0 | 1.8, 22.5 | 5.5, 8.7 | 6.5, 34.1 |
| Day 5: 0.25 Hour | | | | | | |
| Post-Dose | | | | | | |
| Sample Size | 9 | 6 | 6 | 6 | 3 | 6 |
| Mean | 8.77 | 17.28 | 25.28 | 19.25 | 11.80 | 18.98 |
| Standard Deviation | 5.098 | 12.240 | 9.403 | 10.115 | 5.726 | 6.186 |
| Median | 7.00 | 13.05 | 25.05 | 14.85 | 9.60 | 18.75 |
| Min, Max | 3.3, 17.7 | 7.8, 41.0 | 15.1, 40.1 | 9.0, 33.5 | 7.5, 18.3 | 11.4, 29.1 |
| Day 5: 0.5 | | | | | | |
| Hour Post-Dose | | | | | | |
| Sample Size | 9 | 6 | 6 | 6 | 3 | 6 |
| Mean | 7.67 | 20.75 | 32.60 | 20.58 | 23.90 | 17.63 |
| Standard Deviation | 4.123 | 9.873 | 8.017 | 15.668 | 18.975 | 2.960 |
| Median | 6.20 | 17.00 | 32.00 | 14.55 | 14.90 | 17.75 |
| Min, Max | 3.9, 17.4 | 11.4, 37.4 | 20.3, 42.2 | 4.3, 46.3 | 11.1, 45.7 | 12.5, 21.4 |
| Day 5: 1 Hour | | | | | | |
| Post-Dose | | | | | | |
| Sample Size | 9 | 6 | 6 | 6 | 3 | 6 |
| Mean | 9.03 | 16.23 | 30.05 | 22.62 | 27.50 | 15.48 |
| Standard Deviation | 5.458 | 9.989 | 11.433 | 25.506 | 14.001 | 5.998 |
| Median | 7.60 | 11.30 | 26.75 | 10.85 | 27.30 | 14.90 |
| Min, Max | 3.5, 18.0 | 7.0, 30.5 | 17.5, 46.9 | 4.8, 72.4 | 13.6, 41.6 | 9.3, 23.1 |
| Day 5: 1.5 | | | | | | |
| Hours Post-Dose | | | | | | |
| Sample Size | 9 | 6 | 6 | 6 | 3 | 6 |
| Mean | 7.63 | 14.33 | 23.80 | 20.93 | 23.43 | 10.28 |
| Standard Deviation | 3.866 | 7.869 | 10.732 | 25.534 | 7.827 | 4.403 |
| Median | 6.50 | 10.60 | 23.50 | 7.75 | 27.70 | 10.50 |
| Min, Max | 3.8, 15.4 | 8.7, 28.0 | 11.5, 39.5 | 4.5, 69.3 | 14.4, 28.2 | 4.5, 15.9 |
| Day 5: 3 Hours | | | | | | |
| Post-Dose | | | | | | |
| Sample Size | 9 | 6 | 6 | 6 | 3 | 6 |
| Mean | 7.58 | 9.38 | 13.52 | 12.08 | 14.27 | 7.50 |
| Standard Deviation | 4.010 | 5.578 | 4.600 | 11.873 | 3.402 | 2.011 |
| Median | 6.00 | 7.60 | 13.00 | 7.95 | 15.60 | 7.20 |
| Min, Max | 3.7, 16.4 | 4.7, 19.1 | 8.2, 21.3 | 3.4, 35.1 | 10.4, 16.8 | 4.9, 10.9 |
| Day 5: 6 Hours | | | | | | |
| Post-Dose | | | | | | |
| Sample Size | 8 | 6 | 6 | 6 | 3 | 6 |
| Mean | 8.59 | 7.37 | 8.47 | 6.33 | 8.57 | 7.88 |
| Standard Deviation | 3.223 | 4.504 | 2.685 | 2.868 | 3.873 | 3.544 |
| Median | 7.70 | 6.10 | 7.60 | 6.40 | 7.70 | 6.35 |
| Min, Max | 5.5, 15.1 | 2.7, 15.6 | 6.1, 13.7 | 3.0, 9.8 | 5.2, 12.8 | 5.0, 14.0 |
| Day 5: 12 Hours | | | | | | |
| Post-Dose | | | | | | |
| Sample Size | 9 | 6 | 6 | 6 | 3 | 5 |
| Mean | 7.63 | 9.23 | 8.55 | 6.15 | 6.87 | 7.22 |
| Standard Deviation | 2.540 | 4.549 | 2.387 | 2.891 | 2.570 | 4.273 |
| Median | 6.90 | 8.05 | 7.75 | 5.85 | 6.50 | 4.90 |
| Min, Max | 4.0, 12.5 | 5.1, 15.8 | 6.2, 12.7 | 2.9, 9.7 | 4.5, 9.6 | 3.8, 14.2 |
| Day 5: 24 Hours | | | | | | |
| Post-Dose | | | | | | |
| Sample Size | 9 | 6 | 6 | 6 | 3 | 6 |
| Mean | 10.37 | 7.63 | 9.60 | 6.05 | 6.87 | 10.77 |
| Standard Deviation | 5.591 | 4.191 | 3.693 | 3.047 | 1.986 | 8.793 |
| Median | 8.30 | 7.10 | 8.65 | 5.10 | 7.70 | 6.35 |
| Min, Max | 5.1, 20.1 | 3.7, 14.5 | 5.6, 15.1 | 3.0, 10.6 | 4.6, 8.3 | 5.0, 27.5 |

TABLE 8
cGMP Parameters - All subjects

| | | | | | |
|---|---|---|---|---|---|
| Placebo | 3 μg/kg | 4 μg/kg | 4.5 μg/kg | 7 μg/kg | 6 μg/kg |
| (N = 9) | (N = 6) | (N = 6) | (N = 6) | (N = 4) | (N = 6) |

Day 1

| | | | | | | |
|---|---|---|---|---|---|---|
| Cₘₐₓ (ng/mL) | 15.822 | 23.383 | 33.167 | 24.467 | 30.875 | 32.333 |
| Tₘₐₓ (h) | 9.920 | 0.439 | 4.550 | 2.575 | 2.163 | 1.908 |
| AUC₀₋₂₄ (ng*hr/mL) | 244.933 | 202.038 | 248.217 | 210.298 | 198.925 | 283.789 |

Day 5

| | | | | | | |
|---|---|---|---|---|---|---|
| Cₘₐₓ (ng/mL) | 12.811 | 21.400 | 34.733 | 26.350 | 29.600 | 21.983 |
| Tₘₐₓ (h) | 14.341 | 0.472 | 0.725 | 0.494 | 0.861 | 4.461 |
| AUC₀₋₂₄(ng*hr/mL) | 202.991 | 217.994 | 260.591 | 192.386 | 222.326 | 212.430 |

### Example 2—Analysis of Phase 1b Study cGMP Levels with Stratification by Starting Blood Pressure and Total Dose

The results of the Phase 1b study detailed in Example 1 is further analyzed.

Results—cGMP Levels Correlate with Total MANP Dose

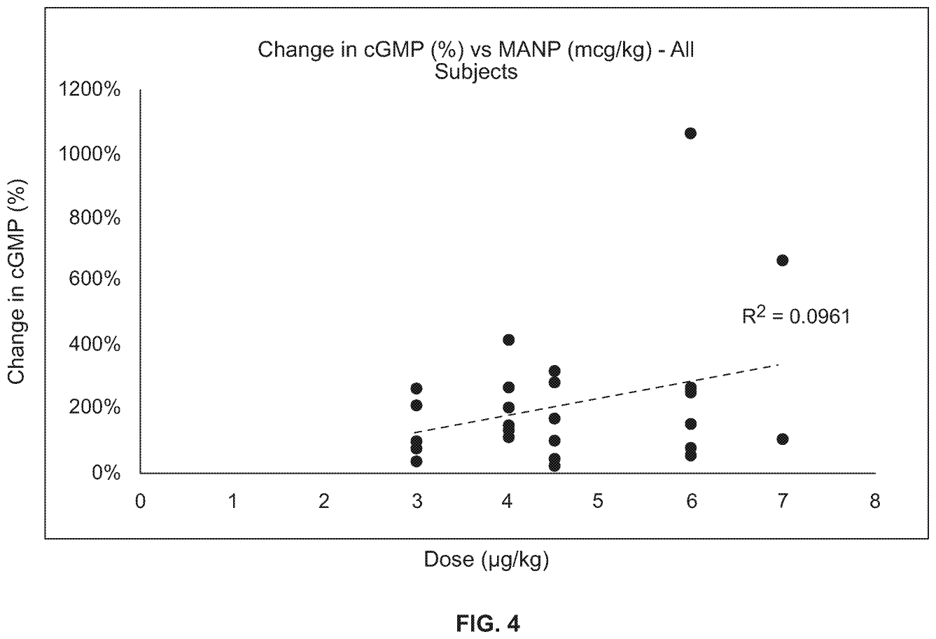

First, it is observed that the change in cGMP levels do not appear to be correlated to the weight-based dose of MANP received (FIG. 4). Other measurements, such as the maximum SBP reduction, show a general weight-based dose-dependence (Table 6). The dependence of cGMP levels with MANP is further investigated.

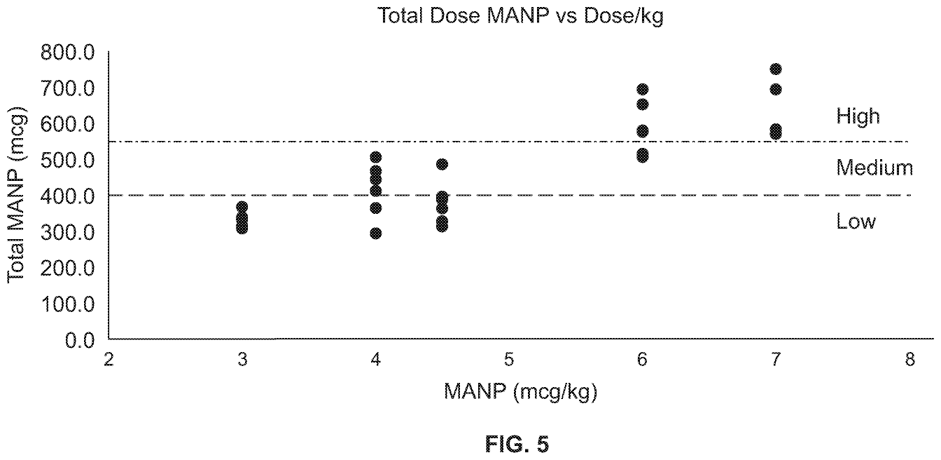

As shown in FIG. 5, while the dose by weight is generally correlated with the total dose of MANP received, there are overlaps in the total dose of MANP received between the different dose groups. For example, some patients in the 4.5 μg/kg dose group received less total MANP than some other patients in the 3.0 μg/kg dose group. Likewise, some patients in the 6.0 μg/kg dose group received approximately the same total amount of MANP as some other patients in the 4.0 μg/kg dose group.

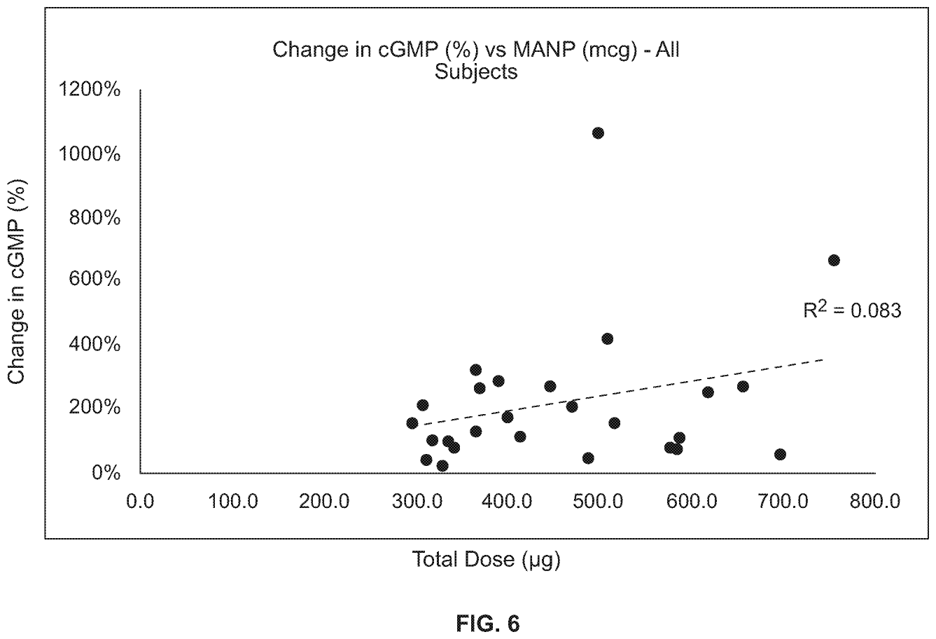

The cGMP data is reanalyzed by grouping the data based on the total amount of MANP received. The patient data is grouped into (i) those who received a low dose (total MANP<400 mcg), a medium dose (400 mcg<total MANP<550 mcg), and a high dose (total MANP>550 mcg). As shown in FIG. 6, change in cGMP levels is correlated to the total dose of MANP. This result supports the use of fixed dosing of MANP.

### Results—Stratification by Starting SBP

A second observation from the Phase 1b study is that, despite the inclusion criteria that patients have a clinic SBP≥140 mmHg (derived from the average BPs from the Screening Visit and Visit 1 readings) and randomization of patients into different dose groups, baseline SBP (derived from Visit 2 Day 1 reading) of the recruited patients differed considerably, not only between dose groups, but also from the inclusion criteria. For example, the 4 μg/kg, 4.5 μg/kg, and the 6 μg/kg dose groups had baseline SBPs of 138.3 mmHg, 134.3 mmHg, and 129.0 mmHg respectively, below the 140 mmHg criteria as measured at the screening visits. Likewise, the baseline SBPS of the different groups ranged from 129.0 mmHg in the 6 μg/kg dose group to 145.9 mmHg in the placebo group.

The unexpected variability in the baseline SBPs provides an opportunity to observe the effects of MANP on patients with SBPs closer to normal. When patients are separated into those with baseline SBP≥140 mmHg or <140 mmHg, a dichotomy emerges in the change in SBP after MANP dosing. As shown in Table 9, MANP appears to lower SBP for patients with baseline SBP≥140 mmHg, but does not appear to have an effect on SBP for patients with baseline SBP<140 mmHg, across all dose groups (low (total MANP<400 mcg), medium (400 mcg<total MANP<550 mcg), and high (total MANP>550 mcg)).

TABLE 9
Change in SBP after MANP dosing - All subjects

SBP Change from Baseline

| | | | | |
|---|---|---|---|---|
| Baseline SBP | Placebo | Low | Medium | High |

Day 1, 2 hours Post-Dose

| | | | | |
|---|---|---|---|---|
| SBP ≥140 mmHg | −8.7 | −17.7 | −8.3 | −46.0 |
| SBP <140 mmHg | 9.3 | 0.0 | −1.5 | 1.6 |

Day 1, 12 hours Post-Dose

| | | | | |
|---|---|---|---|---|
| SBP ≥140 mmHg | −8.8 | −14.4 | −18.7 | −36.2 |
| SBP <140 mmHg | 10.6 | 0.8 | −2.8 | 5.1 |

Day 5, 2 hours Post-Dose

| | | | | |
|---|---|---|---|---|
| SBP ≥140 mmHg | −21.0 | −18.9 | −24.7 | −38.5 |
| SBP <140 mmHg | −8.0 | 0.4 | −4.5 | −0.2 |

Day 5, 12 hours Post-Dose

| | | | | |
|---|---|---|---|---|
| SBP ≥140 mmHg | −18.3 | −16.4 | −19.7 | −37.6 |
| SBP <140 mmHg | −2.2 | −0.7 | −2.8 | 3.5 |

This result shows that the use of MANP, even at high doses, has little risk of causing hypotensive shock or crisis in patients. This result, combined with the rapid lowering of SBP, suggests that MANP may find uses in situations where a fast reduction in BPs is desired, for example in cases of perioperative hypertension, postoperative hypertension, acute severe hypertension, intracerebral hemorrhage, and hypertensive crisis.

Results—cGMP Levels Correlate with Total MANP Dose

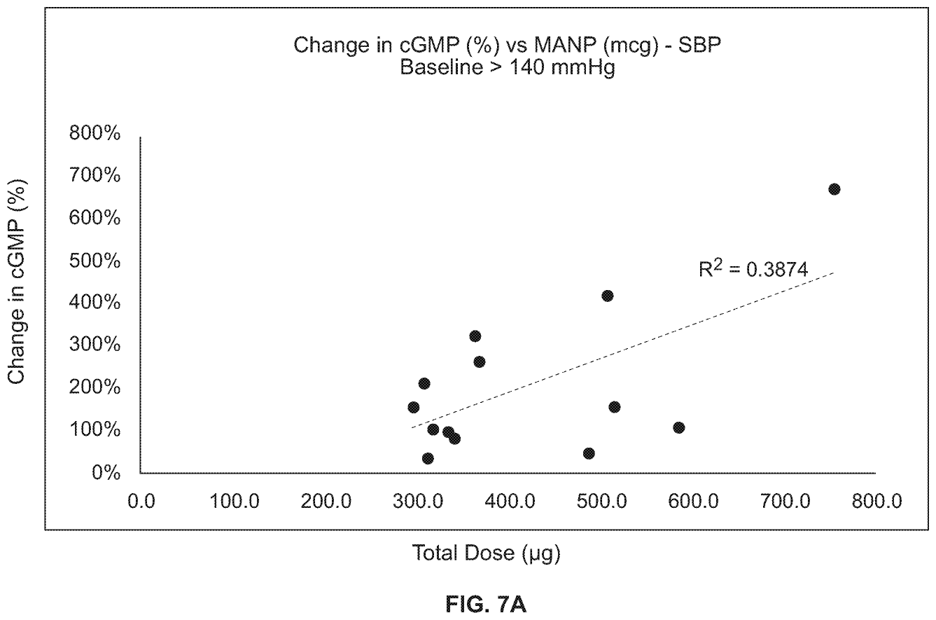

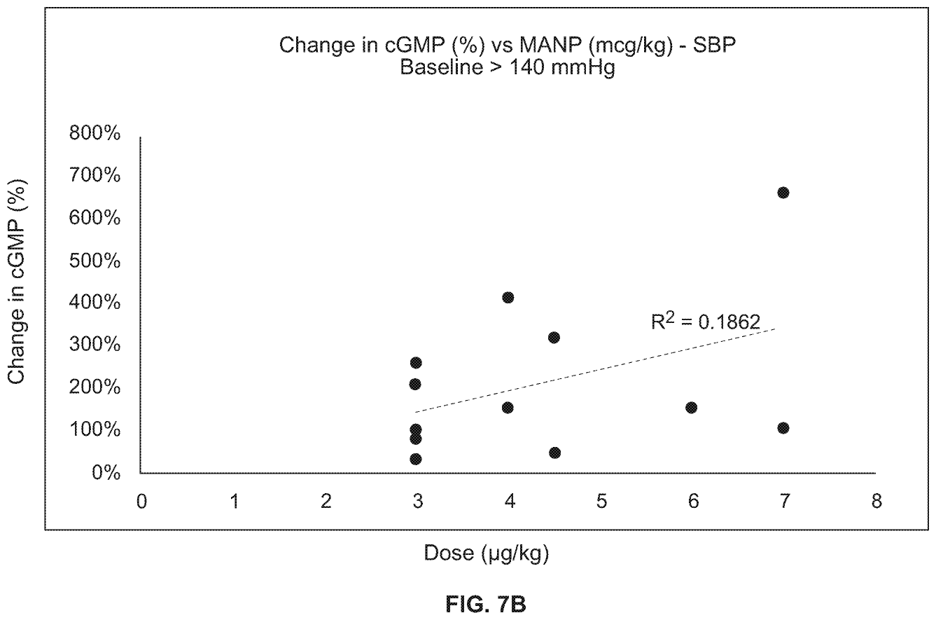

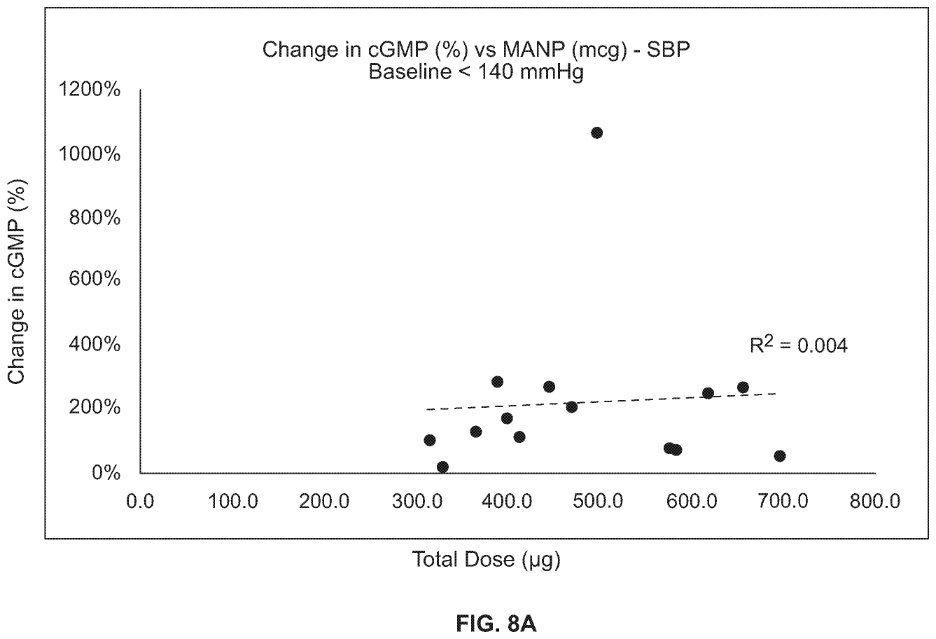

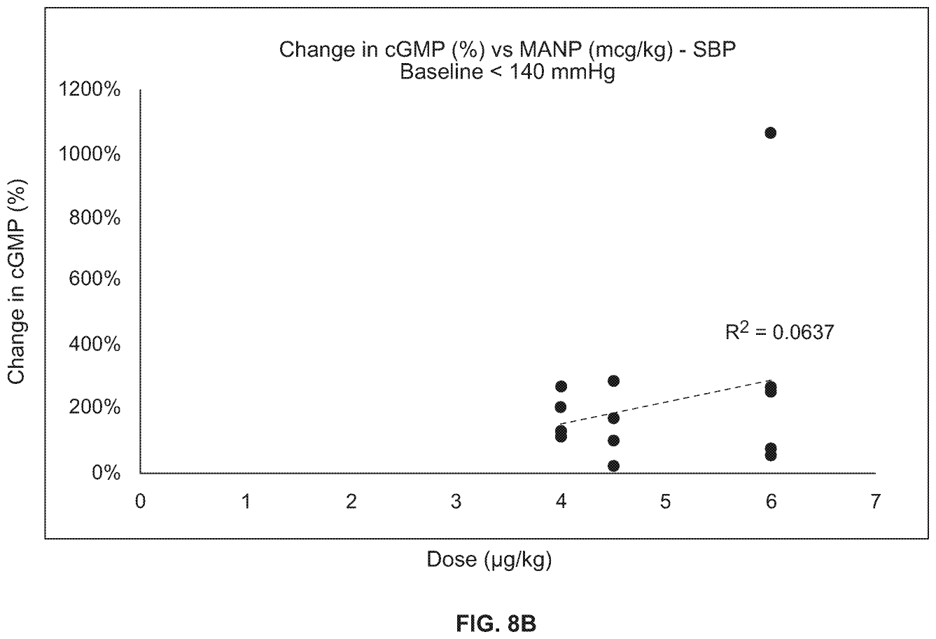

Confirming the results of the earlier cGMP analysis, the correlation becomes more pronounced when the change in cGMP is analyzed by grouping patients with baseline SBP≥140 mmHg and SBP<140 mmHg separately. For patients with SBP≥140, the change in cGMP shows a stronger correlation to the total amount of MANP received (FIG. 7A; R2=0.3874) compared to weight-based doses (FIG. 7B; R2=0.1862). Conversely, for patients with SBP<140, the change in cGMP shows almost no correlation to MANP dose, whether it is based on the total amount of MANP received (FIG. 8A; R2=0.004) or the weight-based dose received (FIG. 8B; R2=0.0637). In all, these results support the use of fixed dosing of MANP.

### Example 3—a Phase 2 Dose-Titration Trial to Evaluate the Safety and Efficacy of MANP in Subjects with Difficult to Control/Resistant Hypertension (BOLD-HTN)

Based on the results of Example 2, a Phase 2 trial dose-titration study using fixed doses is designed and performed. This trial is designed to evaluate the safety and efficacy of once daily MANP by subcutaneous injection compared to placebo in reducing baseline daytime (8 am to 3:59 μm) systolic blood pressure (SBP), derived from 24-hour ambulatory blood pressure monitoring (ABPM), in subjects with hypertension who are taking 3 or more antihypertensive medications with different mechanisms of action.

### Methodology

Subjects on stable doses of three or more hypertensive drugs with different mechanisms of action (including a diuretic and one of an ACE inhibitor or angiotensin receptor blocker (ARB)) prior to screening are selected. Subjects are evaluated for participation in the study during a Screening Period of approximately 14 days, prior to randomization. At the Screening Visit (~ Day-16), patients will have a seated (5 minutes) SBP≥140 mmHg and Daytime SBP≥135 mmHg by ABPM prior to randomization (Visit 1). During the Screening Period, patients will continue taking their current stable doses of prescribed antihypertensive medication. During the Treatment Period, enrolled patients are administered fixed doses of MANP once daily for approximately 42 days, and 24-hour ambulatory blood pressure is performed. During the Treatment Period, patients will continue their background hypertensive medications, with MANP administered no less than one hour after any of the hypertensive medications are taken. At the end of the study, patients are monitored during a follow-up period of approximately 28 days, with follow-up visits on Days 49 and 70.

Approximately 120 adult male and female subjects between the ages of 18 and 80 years with difficult to control hypertension or resistant hypertension (DTC/RH) are planned to be analyzed, with patients randomized 1:1 into two arms (approximately 60 patients each in the placebo and treatment groups). The study will aim to enroll up to 50% African Americans.

After randomization on the morning of Visit 1/Day 1, participants receive a single subcutaneous bolus injection of either MANP or matching placebo once daily in the morning for 42 days at approximately 8 am (±2 hours). The injections will be administered in a rotational manner across the four quadrants of the abdomen and one in each thigh, ensuring that no site receives more than one injection before all sites have been injected. Injection site reaction evaluations will be at 1 hour (±10 minutes) post-dose at every clinic visit. The treatment assignments were double-blind for the duration of the study.

Participants randomized to MANP and placebo will undergo dose escalation, beginning at 300 μg, with subsequent sequential titrations every week at the clinic to 450 μg and maximally to 600 μg based on hypotension and/or orthostatic hypotension symptoms and SBP levels as described in the dosing algorithm below. The dosing algorithm will be reviewed weekly at every scheduled clinic visit and as needed at unscheduled visits.

In addition to evaluating the participant's hydration, serum sodium, seated and standing blood pressure levels, the dosing algorithm relies on assessing any symptoms for hypotension and/or orthostatic hypotension. Evaluation, and if necessary, the correction of dehydration as well as encouraging good hydration habits are important elements of evaluating hypotension and/or orthostatic hypotension. Significant symptomatic hypotension and/or orthostatic hypotension is defined as follows: lightheadedness, dizziness, or visual symptoms with or without standing, accompanied by at least one of the following: (i) seated (5 minutes) SBP<100 mmHg; (ii) a decrease of >20 mmHg between the seated and standing clinic SBP; (iii) a standing SBP<80 mmHg; or (iv) a decrease of seated SBP of ≥35 mmHg from the prior visit.

### Dosing Algorithm:

1. If the clinic seated SBP>130 mmHg and the participant is asymptomatic for hypotension or orthostatic hypotension, the dose is escalated to the next highest dose of study drug.
2. If the participant has symptomatic hypotension and/or orthostatic hypotension and the clinic seated SBP is >130 mmHg, the dose of study drug is maintained until next clinic visit unless the Investigator has safety reservations in which case the study drug can be down-titrated one level. Participants will be instructed to maintain good hydration.
3. If SBP is between 100-130 mmHg and the participant is asymptomatic for hypotension or orthostatic hypotension, the current dose is maintained, and the participant continues receiving this dose for next 7 days (±/−1 day), until the next clinic evaluation.
4. If the participant has significant symptomatic hypotension and/or orthostatic hypotension at SBPs between 100-130 mmHg, the participant temporarily interrupts the study drug and is reevaluated at the clinic within 48-72 hours. If the BP and hypotension and/or orthostatic hypotension symptoms do not stabilize, the participant will discontinue study treatment permanently and begins the early termination (ET) procedures to exit the study. If the BPs and hypotension and/or orthostatic hypotension symptoms stabilize in 48-72 hours after interruption of study drug, the participant may be restarted at a lower previously tolerated dose of study drug and is monitored closely. If this occurs at the starting dose of 300 μg, the dose is reduced to 200 μg.
5. If the SBP is less than 100 mmHg with or without hypotension and/or orthostatic hypotension symptoms, the study drug is paused for 48-72 hours, after which, if any prior symptoms of hypotension and/or orthostatic hypotension resolve, the participant may be restarted at a lower previously tolerated dose of study drug and be monitored closely. If this occurs at the starting dose of 300 μg, the dose is reduced to 200 μg. Alternatively, if the Investigator has safety reservations about restarting the study drug, the participant will discontinue study treatment permanently and begin the early termination (ET) procedures to exit the study.

### Inclusion Criteria:

Patients are eligible for enrollment in the study only if they meet all the following criteria at time of Screening:
1. Patient is willing to provide written informed consent to participate in the study after reading the informed consent form and the information provided and has had the opportunity to discuss the study with the investigator or designee.
2. Patient is aged 18-80 years, inclusive, at the Screening Visit.
3. Patient of childbearing potential will have a negative serum pregnancy test result at Screening and a negative urine pregnancy test on Visit 1/Day 1.
a. Patient who are not of childbearing potential are defined as: (i) Postmenopausal (defined as at least 12 months with no menses in women>45 years of age) with documented blood follicle stimulating hormone (FSH)>40 mIU/mL; OR (ii) Has had a hysterectomy and/or bilateral oophorectomy, bilateral salpingectomy, or bilateral tubal ligation/occlusion at least 3 months prior to randomization; OR (iii) Has had a congenital or acquired condition that prevents childbearing.
b. Patient of childbearing potential agree to avoid becoming pregnant while receiving study treatment and for 14 days after the last study visit by complying with one of the following: (i) Practices abstinence from heterosexual activity; OR (ii) Uses (or have her partner use) acceptable contraception during heterosexual activity. Acceptable methods of contraception are: (a) Hormonal contraception (contraceptive pill or injection) PLUS an additional barrier method of contraception such as a diaphragm, condom, sponge, or spermicide; (b) Vasectomy of a female participant's male partner; (c) In the absence of hormonal contraception or vasectomy of a female participant's male partner, combination contraceptive methods (requiring use of two of the following) must be used: (1) Non-hormonal intrauterine device (IUD); (2) Diaphragm with spermicide (cannot be used in conjunction with cervical cap/spermicide); (3) Cervical cap with spermicide (nulliparous women only); (4) Contraceptive sponge (nulliparous women only); (5) Male condom or female condom (cannot be used together).
4. Patient assigned the male sex at birth is willing to use a barrier birth control method (condom) for ANY sexual activity throughout the study and for 90 days after the last dose of study drug or be at least 6 weeks post-vasectomy with confirmation by post-vasectomy semen analysis. In addition, patient may not donate sperm for the duration of the study and for 90 days after the last dose of study drug.
5. Patient is taking appropriate doses of 3 or more anti-hypertensive medications with different mechanisms of action, one of which must include a diuretic, and if used, ACE inhibitors or ARB doses must be at least 50% of the maximum recommended daily dose for hypertension.
6. Doses and dosing regimen of anti-hypertensive medications are stable for a minimum of 30 days prior to Screening Visit and are not changed during the duration of the study (Screening through the Follow-Up Visit 2) except as described in dosing algorithm.
7. Patient achieves a minimum of 80% overall compliance with the prescribed background antihypertensive medication doses during the period between the Screening Visit and Day 1.
8. Patient has a seated (5 minutes) SBP≥140 mmHg and a daytime mean SBP≥135 mmHg by ABPM prior to randomization (Visit 1).
9. Upon standing BP assessment, the decrease in SBP must be <20 mmHg and the increase in heart rate must be <10 bpm compared to seated BP.
10. Patient has a CKD-EPI eGFR≥30 mL/min/1.73 m². A subset of the participants may have an eGFR between 20-29 mL/min/1.73 m², but they do not exceed 10% of the total study participants.
11. Patient has a BMI between 18-40 kg/m² (inclusive).
12. Patient has arm circumferences of 18-50 cm to accommodate the ABPM cuff.
13. Patient is able to communicate satisfactorily with the investigators and to participate in, and comply with, the requirements of the study.
14. Patient is able to understand the nature of the study and any potential hazards associated with participating in it.

### Exclusion Criteria:

Subjects who meet any of the following criteria are excluded from the study:
1. Has an average sitting systolic blood pressure ≥180 mmHg or diastolic blood pressure >110 mmHg at Screening Visit, or prior to randomization at T1.
2. Has a) Serum sodium of <133 mEq/L or >150 mEq/L; b) Serum potassium of <3.5 mEq/L or >5.5 mEq/L. At the Investigator's discretion, one re-draw is permitted for values inconsistent with the participant's history or the Investigator's clinical assessment.
3. Has a history of secondary hypertension, including but not limited to coarctation of the aorta, primary hyperaldosteronism, renal artery stenosis, Cushing's disease, pheochromocytoma, and polycystic kidney disease. If the subject has not previously been evaluated for secondary hypertension, investigators are responsible for evaluating all potential secondary causes of hypertension in accordance with current practices and clinical guidelines before entering the patient into the study.
4. Has an HbA1c>8% at Screening Visit. At the Investigator's discretion, one re-draw is permitted for values inconsistent with the subject's history or the Investigator's clinical assessment.
5. Has a serum ALT or AST>2×ULN, or Bilirubin (total)>1.5×ULN at Screening Visit or prior to randomization (Visit 1). At the Investigator's discretion, one re-draw is permitted for values inconsistent with the subject's history or the Investigator's clinical assessment.
6. Has experienced myocardial infarction, unstable angina, or a cerebrovascular accident (CVA) within 6 months of the Screening Visit; or sick sinus syndrome or second- or third-degree atrioventricular block, or recurrent atrial tachyarrhythmia, recurrent ventricular tachycardia, or symptomatic bradycardia.
7. Has chronic persistent atrial fibrillation or flutter.
8. Has an implanted cardioverter defibrillator (ICD) that has fired for any arrhythmia within 3 months of Screening Visit or implanted pacemakers.
9. Has congestive heart failure (New York Heart Association [NYHA] class II-IV).
10. Has hemodynamically significant valvular heart disease.
11. Undergoes hemodialysis or peritoneal dialysis, or history of renal transplant.
12. Has diagnosis or recurrence of malignancy within the past 3 years with the exception of successfully treated basal cell or squamous cell carcinoma of the skin.
13. Has sleep apnea and is using or has a prescription to use CPAP therapy.
14. Previously tested positive for HIV antibodies, hepatitis B surface antigen (hBsAg), or hepatitis C virus (HCV) antibodies.
15. Has a history of (within 12 months prior to Screening) or active alcohol abuse. (Note: Alcohol abuse includes heavy alcohol intake as defined by >3 drinks per day or >14 drinks per week or binge drinking).
16. Has a history (within the last 2 years) of, illicit drug use, significant mental illness, physical dependence to any opioid, or any history of drug abuse or addiction.
17. Has a positive urine drug screen including ethanol, cocaine, THC, barbiturates, amphetamines, benzodiazepines, cotinine, and opiates unless, in the opinion of the Investigator, a positive finding results from a legitimate medical prescription for a valid medical condition.
Occasional intermittent use of cannabinoid products will be allowed provided that no cannabinoid products have been used during the 1 week prior to the Screening Visit. The use of cannabinoid products is not permitted after the Screening Visit and for the duration of the study.
18. Has current nicotine use (including smoking, chewing tobacco, and vapor-based products).
19. Receives live or non-live vaccine within 30 days prior to the first dose of study drug or an intention to receive such a vaccine at any time during the study.
20. Performs shift or night work.
21. Has participated in a clinical study involving another investigational drug or device within 1 month or 5 half-lives of the previous Investigational Product (IP) (whichever is longer) prior to the Screening Visit.
22. Requires treatment from Screening Visit to the end of the study with any drug that, in the discretion of the investigator, may affect the assessment of blood pressure in this study or contribute to drug-induced hypertension, including, but not limited to: (a) MAOi drugs, tricyclics, venlafaxine, duloxetine, and bupropion; (b) Oral nitrates; (c) ADHD drugs (e.g., amphetamine); (d) Thyroid medication, estrogen-based and/or androgen-based hormone replacement therapies unless on a stable regimen for at least 3 months prior to Screening Visit; (e) Chronic administration (>3 days/week) of any of the following: (i) Sympathomimetic drugs (e.g., oral/nasal decongestants); (ii) Phosphodiesterase-5 inhibitor (e.g., sildenafil, vardenafil); (iii) Oral corticosteroid treatment; or (iv) NSAIDs or COX-2 inhibitors.

Blood Pressure:

At the Screening Visit, both seated and standing SBPs are measured. Seated SBP is measured as the average of the last two of three seated BPs measured using a validated digital automated blood pressure monitor after the patient has been at rest in the seated position with both feet flat on the floor for at least 5 minutes and with the cuff remaining in position but completely deflated for at least a full minute between measurements. On the other hand, standing SBPs are measured after a patient has been seated for at least 3 minutes. The participants will then stand, and a single blood pressure measurement will be taken 2 minutes after standing. Seated and standing SBPs are also measured in the clinic on Days 1, 7, 14, 21, 28, 35, and 42.

During the study, patients are provided with an ABPM monitoring system. Study personnel will initiate collection of 24-hour ABPM at approximately 8 am on Days 1, 20, 27, and 41. For each ABPM, data collection is ended only after a full 24-hours of monitoring. On treatment non-clinic days (Visit 1 through Visit 7), participants will take two blinded BP readings using the blinded home BP monitor, approximately 1 hour before self-administering the MANP injection.

### Primary Outcome Measure:

For the duration of the study, ABPM of patients are measured using a ABPM monitor. A primary outcome measure is the change from baseline in mean daytime SBP (8:00 am to 3:59 μm) derived from 24-hour ABPM at approximately Day 42 of trial.

Other primary outcome measures for safety will also be evaluated at approximately 10 weeks after the first dose, including: (1) incidence and severity of adverse events (AE) through 4-weeks post end of treatment; (2) incidence and severity of serious adverse events (SAE) through 4-weeks post end of treatment; (3) incidence and severity of treatment emergent adverse events (TEAE) through 4-weeks post end of treatment; (4) change/shifts in laboratory values from baseline through 4-weeks after the end of treatment; (5) change in vital signs including BP and heart rate from baseline through 4 weeks after the end of treatment; (6) change in ECG parameters compared to baseline at 42 days post-dose or at end of treatment visit for early termination participants.

### Secondary Outcome Measures

For the duration of the study, the following secondary outcomes are also measured:
1. Change from baseline in mean daytime DBP and heart rate derived from 24-hour ABPM at the last visit (approximately Day 42).
2. Change from baseline in mean 24-hour and night-time SBP, DBP, and heart rate derived from 24-hour ABPM at the last visit (approximately Day 42).
3. Change from baseline in mean pre-dose seated SBP, DBP, and heart rate at the last visit (approximately Day 42).
4. Change from baseline in mean ABPM daytime, 24-hour, nighttime SBP, DBP, heart rate, and pre-dose seated SBP at approximately Day 21 (mid-study timepoint).
5. Achieving goal mean daytime SBP≤130 mmHg from ABPM at last visit.
6. Achieving mean seated (5 minutes) SBP control≤130 mmHg at last visit.
7. Achieving goal mean daytime SBP≤130 mmHg from ABPM at mid-study.
8. Achieving mean seated (5 minutes) SBP control≤130 mmHg at mid-study.
9. Blood samples will be collected for MANP and cGMP concentration measurements at Baseline, at each clinical visit, and Follow-up (FU) Visits 1 and 2. The following PK parameters, but limited to, will be calculated, where applicable: (a) MANP and cGMP at Visit 1/Day 1 and Visit 7: pre-dose (−20 min), and at 5 min, 15 min, 30 min, 60 min, 90 min, 180 min, and 6 hours post-dose; (b) MANP and cGMP at Visits 2, 3, 4, 5, 6: pre-dose (−20 min) and 60 min post-dose; (c) MANP and cGMP at Follow-Up Visit 1: at any time-point; (d) cGMP only at Follow-Up Visit 2: at any time-point; (e) Maximum observed plasma concentrations (Cₘₐₓ) at Visit 1 and Visit 7; (f) Time (Tₘₐₓ) at which Cₘₐₓ is attained at Visits 1 and 7; (g) Area under the plasma concentration versus time curve from time zero to time of last observed quantifiable concentration (AUC₀₋ₜₗₐₛₜ) and AUC_{0-infinity} at Visits 1 and 7; (h) Trough concentrations of MANP at all visits as applicable; and (i) Change from baseline in plasma cGMP at Follow-Up Visit 1 (~1-week after end of treatment) and Follow-Up Visit 2 (~ 4 weeks after end of treatment).
10. Change in plasma anti-MANP antibodies from baseline to Day 21 (mid-study visit), Day 42 (Visit 7), Follow-Up Visit 1 (Day 49), and to Follow-Up Visit 2 (Day 70) (~4 weeks after the end of treatment).
11. Change in plasma anti-ANP antibodies from baseline to Day 21 (mid-study visit), Day 42 (Visit 7), Follow-Up Visit 1 (Day 49), and to Follow-Up Visit 2 (Day 70) (~4 weeks after the end of treatment).

### Other Outcome Measures

For the duration of the study, the following outcomes are also measured:
1. Change from baseline in metabolic biomarkers in blood (including plasma glucose, HbA1c, C-Peptide, and insulin levels) at the end of treatment and FU 2 (~4-weeks after the end of treatment).
2. Change in baseline lipid biomarkers in blood (including HDL, triglycerides, LDL, Cholesterol, LPa) at end of treatment and FU 1 (~1-week after the end of treatment).
3. Change from baseline in insulin resistance as measured by HOMA-IR at FU 1 (~1-week after the end of treatment) and FU 2 (~4 weeks after the end of treatment).
4. Change from baseline in weight and waist circumference measurements throughout the study, FU 1 (~1-week after the end of treatment), and FU 2 (~4 weeks after the end of treatment).
5. Change from baseline in N-terminal pro b-type natriuretic peptide (NT-proBNP) in blood at end of treatment visit and FU 1 (~1-week after the end of treatment) and aldosterone.
6. Change from baseline in estimated glomerular filtration rate (eGFR), circulating ANP levels, urine albumin, serum creatinine, and cystatin C, at end of treatment visit.
7. Urinary cGMP, sodium, potassium and creatinine clearance at Visits 1 and 7.
8. Change in mean daytime SBP and ABPM from baseline compared to end of treatment visit in AA population and the (NAA) population at each dose level.
9. Difference between AA population and NAA population in change in dose required to reach goal blood pressure.
10. Changes in the following parameters from run-in (baseline) through treatment and follow-up through the use of a wearable ECG patch: (a) Atrial Fibrillation Burden; (b) Heart rate and heart rate variability; (c) Daily gross activity; (d) Daily step counts; (e) Daily walking minutes.
11. Compare the use of blinded home BP measurements with clinic BP measurements in evaluating dose titrations.

From the foregoing, it will be appreciated that the present invention can be embodied in various ways, which include but are not limited to the following:

### LIST OF EMBODIMENTS

Embodiment 1. A method of treating resistant hypertension in a patient in need thereof, comprising administering to the patient about 300 mcg of MANP.

Embodiment 2. A method of treating resistant hypertension in a patient in need thereof, comprising administering to the patient about 450 mcg of MANP.

Embodiment 3. A method of treating resistant hypertension in a patient in need thereof, comprising administering to the patient about 600 mcg of MANP.

Embodiment 4. The method of any one of Embodiments 1-3, wherein the MANP is administered once every 24 hours.

Embodiment 5. The method of any one of Embodiments 1-4, wherein the patient has a systolic blood pressure of greater than or equal to about 140 mmHg prior to the administering.

Embodiment 6. The method of any one of Embodiments s 1-5, wherein the MANP is a peptide having an amino acid sequence consisting essentially of SEQ ID NO:1.

Embodiment 7. The method of any one of Embodiments 1-6, wherein the patient is taking 3 or more anti-hypertensive medications prior to the administering.

Embodiment 8. The method of any one of Embodiments 1-7, wherein the patient has a CKD-EPI eGFR between 20-29 mL/min/1.73 m² prior to the administering.

Embodiment 9. The method of any one of Embodiments 1-7, wherein the patient has CKD-EPI eGFR greater than or equal to 30 mL/min/1.73 m prior to the administering.

Embodiment 10. The method of any one of Embodiments 1-9, wherein the patient has a body-mass index (BMI) of greater than 18 kg/m² prior to the administering.

Embodiment 11. The method of Embodiment 10, wherein the patient has a body-mass index (BMI) of no more than 40 kg/m² prior to the administering.

Embodiment 12. The methods of any one of Embodiments 1-11, further comprising administering one or more anti-hypertensive medications.

Embodiment 13. A method of lowering systolic blood pressure in a patient in need thereof, comprising administering to the patient about 300 mcg of MANP.

Embodiment 14. A method of lowering systolic blood pressure in a patient in need thereof, comprising administering to the patient about 450 mcg of MANP.

Embodiment 15. A method of lowering systolic blood pressure in a patient in need thereof, comprising administering to the patient about 600 mcg of MANP.

Embodiment 16. The method of any one of Embodiments 13-15, wherein the MANP is administered once every 24 hours.

Embodiment 17. The method of any one of Embodiments 13-16, wherein the patient has a systolic blood pressure of greater than or equal to about 140 mmHg prior to the administering.

Embodiment 18. The method of any one of Embodiments 13-17, wherein the MANP is a peptide having an amino acid sequence consisting essentially of SEQ ID NO:1.

Embodiment 19. The method of any one of Embodiments 13-18, wherein the patient is taking 3 or more hypertensive medications prior to the administering.

Embodiment 20. The method of any one of Embodiments 13-19, wherein the patient has eGFR between 20-29 mL/min/1.73 m² prior to the administering.

Embodiment 21. The method of any one of Embodiments 13-20, wherein the patient has eGFR greater or equal to 30 mL/min/1.73 m² prior to the administering.

Embodiment 22. The method of any one of Embodiments 13-21, wherein the patient has a body-mass index (BMI) of 18 kg/m² to 40 kg/m² prior to the administering.

Embodiment 23. The method of any one of Embodiments 13-22, wherein the patient in need of lowering systolic blood pressure has a condition selected from the group consisting of perioperative hypertension, postoperative hypertension, acute severe hypertension, intracerebral hemorrhage, hypertensive emergency, and hypertensive crisis.

Embodiment 24. The method of any one of Embodiments 13-23, wherein the patient has a systolic blood pressure of at least 80 mmHg after the administering.

Embodiment 25. The method of any one of Embodiments 13-24, wherein the patient has a systolic blood pressure of at least 10% lower after the administering compared to prior to the administering.

While the invention has been described with reference to particular aspects, it will be understood by those skilled in the art that various changes may be made and equivalents may be substituted for elements thereof without departing from the scope of the invention. In addition, many modifications may be made to a particular situation or material to the teachings of the invention without departing from the scope of the invention. Therefore, it is intended that the invention not be limited to the particular aspects disclosed but that the invention will include all aspects falling within the scope and spirit of the appended claims.

We claim:

1. A method of treating resistant hypertension in a patient in need thereof, comprising administering to the patient about 300 mcg of MANP.

2. A method of treating resistant hypertension in a patient in need thereof, comprising administering to the patient about 450 mcg of MANP.

3. A method of treating resistant hypertension in a patient in need thereof, comprising administering to the patient about 600 mcg of MANP.

4. The method of any one of claims 1-3, wherein the MANP is administered once every 24 hours.

5. The method of any one of claims 1-4, wherein the patient has a systolic blood pressure of greater than or equal to about 140 mmHg prior to the administering.

6. The method of any one of claims 1-5, wherein the MANP is a peptide having an amino acid sequence consisting essentially of SEQ ID NO:1.

7. The method of any one of claims 1-6, wherein the patient is taking 3 or more anti-hypertensive medications prior to the administering.

8. The method of any one of claims 1-7, wherein the patient has a CKD-EPI eGFR between 20-29 mL/min/1.73 m² prior to the administering.

9. The method of any one of claims 1-7, wherein the patient has CKD-EPI eGFR greater than or equal to 30 mL/min/1.73 m prior to the administering.

10. The method of any one of claims 1-9, wherein the patient has a body-mass index (BMI) of greater than 18 kg/m² prior to the administering.

11. The method of claim 10, wherein the patient has a body-mass index (BMI) of no more than 40 kg/m² prior to the administering.

12. The methods of any one of claims 1-11, further comprising administering one or more anti-hypertensive medications.

13. A method of lowering systolic blood pressure in a patient in need thereof, comprising administering to the patient about 300 mcg of MANP.

14. A method of lowering systolic blood pressure in a patient in need thereof, comprising administering to the patient about 450 mcg of MANP.

15. A method of lowering systolic blood pressure in a patient in need thereof, comprising administering to the patient about 600 mcg of MANP.

16. The method of any one of claims 13-15, wherein the MANP is administered once every 24 hours.

17. The method of any one of claims 13-16, wherein the patient has a systolic blood pressure of greater than or equal to about 140 mmHg prior to the administering.

18. The method of any one of claims 13-17, wherein the MANP is a peptide having an amino acid sequence consisting essentially of SEQ ID NO:1.

19. The method of any one of claims 13-18, wherein the patient is taking 3 or more hypertensive medications prior to the administering.

20. The method of any one of claims 13-19, wherein the patient has eGFR between 20-29 mL/min/1.73 m² prior to the administering.

21. The method of any one of claims 13-20, wherein the patient has eGFR greater or equal to 30 mL/min/1.73 m² prior to the administering.

22. The method of any one of claims 13-21, wherein the patient has a body-mass index (BMI) of 18 kg/m² to 40 kg/m² prior to the administering.

23. The method of any one of claims 13-22, wherein the patient in need of lowering systolic blood pressure has a condition selected from the group consisting of perioperative hypertension, postoperative hypertension, acute severe hypertension, intracerebral hemorrhage, hypertensive emergency, and hypertensive crisis.

24. The method of any one of claims 13-23, wherein the patient has a systolic blood pressure of at least 80 mmHg after the administering.

25. The method of any one of claims 13-24, wherein the patient has a systolic blood pressure of at least 10% lower after the administering compared to prior to the administering.
